(19) United States
(12) Patent Application Publication
(10) Pub. No.: US 2012/0122957 A1
(43) Pub. Date: May 17, 2012

(54) REGULATION OF AGING BY MODULATION OF MITOCHONDRIAL FUNCTION

(75) Inventors: Andrews Dillin, La Jolla, CA (US); Jennie Durieux, La Jolla, CA (US)

(73) Assignee: The Salk Institute for Biological Studies, La Jolla, CA (US)

(21) Appl. No.: 13/264,939

(22) PCT Filed: Apr. 19, 2010
(86) PCT No.: PCT/US2010/031641

§ 371 Date: Feb. 2, 2012

Related U.S. Application Data
(60) Provisional application No. 61/170,518, filed on Apr. 17, 2009.
(60) Provisional application No. 61/299,800, filed on Jan. 29, 2010.

(51) Int. Cl. A61K 31/713; A61P 43/00; G01N 33/48

(52) U.S. Cl. 514/44 A; 800/3

(57) ABSTRACT

The invention relates to the field of longevity enhancement. More particularly, the invention provides compositions and methods relating to modulation of mitochondrial function. In certain embodiments, the invention provides methods and related compositions for the enhancement of longevity in an animal, comprising inhibition of one or more electron transport chain components, such as cco-1 and homologs thereof, in a tissue-specific manner in the animal.

### CROSS-REFERENCE TO RELATED APPLICATIONS

This application claims the benefit of priority of U.S. provisional application Ser. No. 61/170,518, filed Apr. 17, 2009, and U.S. provisional application Ser. No. 61/299,800, filed Jan. 29, 2010.

The foregoing applications, and all documents cited therein, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTIONS

The invention relates to the field of longevity enhancement. More particularly, the invention provides compositions and methods relating to modulation of mitochondrial function.

### BACKGROUND OF THE INVENTION

An aging organism exhibits correlated, recognizable, and predictable changes to its physiology over time. These changes occur coordinately across multiple tissues and organs, in concordance with theories that posit a strong role for the participation of the endocrine system in the regulation of age-related phenotypes (Russell and Kahn, 2007; Tatar et al., 2003). Within invertebrate model organisms such as C. elegans and Drosophila, evidence strongly suggests that tissue-specific manipulations of endocrine pathway components affect the aging process of the entire organism. These include alteration of signals from the somatic germline which control the aging of non-mitotic tissues (Hsin 1999; Arrantes-Oliveira 2002); restoration or reduction of insulin/IGF-1 signaling (IIS) in neuronal or fat tissues (Broughton et al., 2005; Hwangbo et al., 2004; Kapahi et al., 2004; Libina et al., 2003; Wolkow et al., 2000); and genetic manipulations to specific neurons which then alter the capacity for the entire animal to respond to dietary restriction (Bishop and Guarente 2007). These systems have offered the simplicity of studying tissue-specific expression in organisms in which single-gene mutations can affect longevity. These findings have been extended to mammalian model systems. In mice, for example, fat-specific knock out of the insulin receptor extends lifespan (Bluher et al., 2003) and loss of neuronal IRS2, one of the insulin receptor substrates, results in increased longevity in mice (Taguchi et al., 2007). Furthermore, parabiotic pairings between young and old mice restores young phenotypes in aged progenitor cells (Conboy et al., 2005). Such evidence strongly suggests that there are key tissues that transmit longevity signals to additional tissues to regulate the aging process. Moreover, these adaptations may have evolved to provide the animal with a mechanism by which an environmental, extrinsic signal could be sensed and then amplified across the entire animal to coordinate the appropriate onset of reproduction, senescence and/or aging.

The regulation of aging is extraordinarily complex; cells, tissues, and even organs can age autonomously within the same individual (Apfeld and Kenyon, 1998; Wessells et al., 2004b). For example, C. elegans muscle undergoes extensive deterioration (sarcopenia) with age, but neurons remain pristine (Herndon et al., 2002). Within the hypodermis (skin) of C. elegans, several cells have a tendency to disappear with age, while others remain functioning (Golden et al., 2007), and recent data suggests that the apparent organism-wide coordination of aging might really reflect subtle, additive changes to levels in cell-autonomous signals (Iser et al., 2007). Even more dramatically, a stress-resistant heart can beat within a fly that is otherwise aging normally (Wessells et al., 2004a). This raises the question whether all interventions affecting aging and lifespan work via hormonal signals, or whether some of the aging processes are regulated in a cell-autonomous fashion.

One of the best-studied manipulations by which lifespan can be increased is via reduced function of the mitochondria. Mutation or reduced function in nuclear genes encoding electron transport chain (ETC) components in yeast, C. elegans, Drosophila, and mice delay the aging process (Copeland et al., 2009; Dell'Agnello et al., 2007; Dillin et al., 2002b; Feng et al., 2001; Hansen et al., 2008; Kirchman et al., 1999; Lapointe et al., 2009; Lee et al., 2002; Liu et al., 2005). Manipulations that impair mitochondria function hold no obvious relationship to well-characterized aging-related changes in insulin signaling, and work in a signaling pathway independent both temporally and genetically from those which regulate aging via nutrient sensing (Dillin et al., 2002b; Feng et al., 2001; Giannakou et al., 2008; Hwangbo et al., 2004; Lee et al., 2002; Wolff et al., 2006; Wong et al., 1995). The insulin/IGF-1 signaling (IIS) pathway is conserved from worms to humans that results in increased longevity when reduced, requiring the forkhead transcription factor, DAF-16/FOXO (Kenyon et al., 1993; Ogg et al., 1997), and co-regulators SMK-1 (Wolff et al., 2006) and HCF-1 (Li et al., 2008). Increased lifespan due to an additional longevity pathway, dietary restriction (DR), is distinct from the IIS pathway as it requires the forkhead transcription factor pha-4/Foxa (Panowski et al., 2007) and the basic-leucine zipper transcription factor skn-1/Nrf2 (Bishop and Guarente, 2007). In contrast, the increased longevity caused by either mutation or RNAi of mitochondrial ETC components does not require daf-16, smk-1, pha-4, or skn-1 (Dillin et al., 2002b; Feng et al., 2001; Lee et al., 2002; Panowski et al., 2007; Rea et al., 2007; Tullet et al., 2008; Wolff et al., 2006). Furthermore, the increased longevity caused by reduced IIS signaling is synergistic with either mutation or RNAi of mitochondrial ETC components (Dillin et al., 2002b; Feng et al., 2001; Lee et al., 2002; Wolff et al., 2006; Wong et al., 1995).

The temporal requirements of the mitochondrial ETC longevity pathway are also distinct from the IIS pathway. In the worm, conditional RNAi experiments revealed that the IIS pathway is required during the reproductive period of adulthood to regulate the aging process (Dillin et al., 2002a; Dillin et al., 2002b). In the fly, the reproductive period of adulthood was also found to be a key time during which the IIS pathway regulates the aging process (Giannakou et al., 2008; Hwangbo et al., 2004). In contrast, reduction of the ETC longevity pathway during adulthood did not result in increased longevity. Using the same conditional RNAi approaches, the larval developmental period proved to be a critical period in which this pathway modulates the aging process (Dillin et al., 2002b). The timing of this pathway was further refined to the L3/L4 stages of larval development (Rea et al., 2007). Thus, the sensing and monitoring of key events during the L3/L4 transition by the ETC longevity pathway initiates and maintains the rate of aging of the animal for the rest of its life.

Citation or identification of any document in this application is not an admission that such document is available as prior art to the present invention.

### SUMMARY OF THE INVENTION

The present invention provides methods of increasing longevity in an animal, comprising modulating electron transport chain function in a tissue specific manner in the animal. In some embodiments, the invention relates to modulating electron transport chain function in intestinal tissue. In other embodiments, the instant invention relates to modulating electron transport chain function in neuronal tissue.

In certain embodiments, the invention relates to modulating the activity of cco-1 or a homolog thereof.

In other embodiments, the instant invention provides methods of increasing longevity in an animal, comprising modulating the mitochondrial unfolded protein response system in the animal.

In certain embodiments, the invention relates to a method of modulating ubl-5 or a homolog thereof.

In yet other embodiments, the invention relates to methods of modulating electron transport chain function in an animal, wherein a cell non-autonomous signal is produced. In some embodiments, the invention relates to methods of modulating the mitochondrial unfolded protein response in an animal, wherein a cell non-autonomous signal is produced.

In certain embodiments, the electron transport chain function is modulated by administration of an inhibitor of an electron transport chain component. In a particular embodiment, the inhibitor is an siRNA. In certain embodiments, the electron transport chain component is cco-1 or a homolog thereof. In further embodiments, the mitochondrial unfolded protein response system is modulated by administration of an inhibitor of a component of the unfolded protein response system. In a particular embodiment, the component is ubl-5 or a homolog thereof.

In yet other embodiments, the invention provides a method of identifying a compound that modulates longevity, comprising contacting a non-human animal with a test compound; and measuring the activity of an electron transport chain (ETC) component in the presence and absence of the test compound in the animal, wherein a test compound that inhibits the activity of the ETC component indicates a compound that modulates longevity. In certain embodiments, the non-human animal is C. elegans.

In some embodiments, the activity of the ETC component that is inhibited is expression of the ETC component in the animal.

In certain embodiments, the electron transport chain component is cco-1 or a homolog thereof. In some embodiments, the non-human animal expresses cco-1 in a tissue-specific manner.

In some embodiments, the test compound enhances longevity in the non-human animal. In certain embodiments, the test compound inhibits expression of cco-1 in intestinal and/or neuronal tissue of the non-human animal.

In some embodiments, the method of identifying a compound that modulates longevity further comprises assessing induction of the mitochondrial unfolded protein response in the animal. In certain embodiments, the test compound induces the mitochondrial unfolded protein response in the non-human animal.

In yet other embodiments, the invention relates to a pharmaceutically acceptable composition comprising a mitokine in an amount effective to stimulate the mitochondrial unfolded protein response in an animal in need thereof. In certain embodiments, the invention relates to a method of enhancing longevity in an animal, comprising administering to the animal a pharmaceutically effective amount of a composition comprising a mitokine in an amount effective to stimulate the mitochondrial unfolded protein response in the animal. In certain embodiments, the animal is a human.

It is noted that in this disclosure and particularly in the claims, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

FIG. 1 shows lifespan analysis of cco-1 hairpin transgenic animals.

A. Wildtype worms allow import of dsRNA from surrounding tissues, but sid-1(qt9) mutant worm can not import dsRNA and RNAi knockdown is no longer systemic but is maintained locally within the tissue in which the dsRNA is produced (Winston et al., 2002)

B. Intestine-specific knockdown of cco-1 results in lifespan extension. sid-1(qt9)/rol-6 control (black line, mean 18.8+/−0.7 days), ges-1p::cco-1hairpin (gray line, 23.9+/−0.8 days, p<0.0001).

C. Body wall muscle knockdown of cco-1 does not significantly effect lifespan. sid-1(qt9)/rol-6 control (black line, mean 18.6+/−0.5 days), myo-3p::cco-1 hairpin (gray line, mean 16.6+/−0.5 days, p=0.0574).

D. Neuronal knockdown of cco-1 extends lifespan. sid-1(qt9)/rol-6 control (black line, 18.2+/−0.2 days), rab-3p::cco-1 hairpin (gray line, 21.7+/−0.5 days, p<0.0001).

E. Neuronal knockdown of cco-1 driven by the unc-119 promoter also extends lifespan. sid-1(qt9)/rol-6 control (black line, mean 19.8+/−0.7 days), unc-119p::cco-1 hairpin (gray line, mean 23.8+/−0.8 days, p=0.0001). Please see Table 1 for all statistical analysis and Table 5 for statistical analysis of additional HP lines.

FIG. 2 shows lifespan analysis of tissue specific complementation of rde-1 with cco-1 feeding RNAi.

A. Tissues exposed to dsRNA from feeding RNAi initiate knockdown if rde-1 has been rescued in the corresponding tissue. Neighboring tissues are unable to initiate RNAi if rde-1 is absent.

B. rde-1(ne219) mutants do not respond to cco-1 feeding RNAi. Animals fed bacteria harboring an empty vector (black line, mean 18.0+/−0.3 days), cco-1 RNAi (gray line, mean 18.16+/−0.4 days, p<0.4043).

C. rde-1 rescued in the intestine (VP303) extends lifespan when fed cco-1 dsRNA producing bacteria. Animals fed vector only bacteria (black line, mean 14.7+/−0.6 days), cco-1 RNAi (gray line, mean 22.0+/−0.2 days, p<0.0001).

D. rde-1 rescued in the body wall muscle (NR350.5) decreases lifespan when fed cco-1 dsRNA bacteria. Animals fed bacteria harboring empty vector (black line, mean 13.5+/−0.3 days), cco-1 RNAi (gray line, mean 11.8+/−0.3 days, p<0.0002.

E. rde-1 rescued in the hypodermis (NR222) has no effect on lifespan when fed cco-1 dsRNA producing bacteria. Vector only (black line, mean 13.5+/−0.3 days), cco-1 RNAi (gray line, mean 14.3+/−0.4 days, p=0.148). Please see Table 1 for all statistical analysis.

FIG. 3 shows induction of the UPR^{mt} is specific to the ETC longevity pathway.

A. hsp-6p::GFP reporter worms fed empty vector (EV) containing bacteria have low levels of background GFP (i) overlay; (ii) GFP. hsp-6p::GFP reporter worms fed cco-1 RNAi upregulate the UPR^{mt}. Relative fluorescence was quantified using a fluorescence plate reader (iii).

B. daf-2(e1370) RNAi does not induce hsp-6p::GFP (i) overlay; (ii) GFP. hsp-6p::GFP reporter worms were hatched on empty vector, cco-1, or daf-2 dsRNA expressing bacteria and allowed to grow to day 1 of adult hood. Relative fluorescence was quantified (iii).

C. Dietary restricted eat-2 (ad1116) mutant worms do not upregulate hsp-6p::GFP reporter (i) overlay; (ii) GFP. Mutant eat-2 worms crossed to hsp-6p::GFP animals did not show GFP induction. Relative fluorescence was quantified (iii).

D. The UPR^{ER} is not induced by cco-1 RNAi, (i) overlay; (ii) GFP. hsp-4-p::GFP transgenic reporter worms were fed empty vector containing bacteria or cco-1 dsRNA bacteria. No fluorescence upregulation was detected (iii). Both EV and to a lesser extent cco-1 RNAi fed worms were able to upregulate the UPR^{ER} upon treatment with tunicamycin, (i and ii) which is known induce UPR^{ER}. Relative fluorescence of was quantified (iii).

E. cco-1 RNAi does not induce a marker of cytosolic protein misfolding stress, (i) overlay; (ii) GFP. hsp16.2p::GFP reporter worms were fed EV or cco-1 dsRNA bacteria. No fluorescence upregulation was detected (iii). As positive controls, heat shock for 6 hours at 31° C. could induce the heat shock response (HSR) and cco-1 RNAi did not block this response (i and ii).

FIG. 4 shows that ubl-5 is necessary and specific for ETC mediated longevity.

A. The long lifespan of isp-1(qm150) mutant animals is dependent upon ubl-5. isp-1(qm150) (empty vector, black line, mean 25.8+/−1.0 days), isp-1(qm150) fed ubl-5 dsRNA bacteria (light gray line, mean 15.5+/−0.7 days, p<0.0001), N2 wildtype (dark gray line, mean 19+/−0.5 days).

B. daf-2(e1370) mutant lifespan is unaffected by ubl-5 knockdown. daf-2(e1370) mutant animals grown on empty vector bacteria (black line, mean 40.1+/−1.2 days), daf-2(e1370) fed ubl-5 dsRNA bacteria (gray line, mean 39.9+/−1.2 days, p=0.327).

C. Dietary restricted eat-2(ad1116) mutant lifespan is not dependent upon ubl-5. N2 on empty vector (dark gray line) mean lifespan 18.2+/−0.4 days; eat-2(ad1116) on empty vector mean 26.4+/−0.6 days; eat-2(ad1116) fed ubl-5 dsRNA bacteria mean 23.3+/−0.7 days, p<0.0004.

D. N2 wildtype lifespan is unaffected by ubl-5 knockdown. N2 grown on empty vector bacteria (black line, mean 18.2+/−0.4 days), N2 fed ubl-5 dsRNA bacteria (gray line, mean 20.3+/−0.4 days, p=0.0834). All statistical data can be found in Table 1.

FIG. 5 shows the temporal activation of ETC generated longevity signal is coincident with induction of the UPR^{mt}.

A. hsp-6p::GFP reporter worms were transferred to cco-1 RNAi at each larval developmental stage and early adulthood. GFP fluorescent measurements were taken 16 hours after reaching young adulthood in all cases.

B. hsp-6p::GFP is upregulated if transfer occurs before the L4 stage of development. C. Quantification of hsp-6p::GFP in (A).

C. Worms transferred as young adults onto cco-1 RNAi can not upregulate the hsp-6p::GFP after being transferred onto cco-1 dsRNA bacteria in adulthood.

D. cco-1 knockdown during larval development is sufficient to induce the hsp-6p::GFP reporter in adulthood. hsp-6p::GFP reporter worms were grown on cco-1 dsRNA bacteria during development and then moved to dcr-1 dsRNA producing bacteria at the L4 larval stage, to disrupt the RNAi machinery allowing CCO-1 levels to return to normal. UPR^{mt} remains induced.

E. hsp-6p::GFP fluorescence was measured 48 hours after transfer to dcr-1 RNAi as described by schematic (D).

FIG. 6 shows Cell non-autonomous upregulation of the UPR^{mt}.

A. Representation of cell autonomous and non-autonomous upregulation of UPR^{mt}. “X's” depict tissue where cco-1 is knocked down (intestine or neurons). Dark gray indicates location of upregulation of hsp-6p::GFP reporter (intestine upon knockdown in intestine or neurons).

B. hsp-6p::GFP reporter worms were crossed to tissue-specific cco-1 hairpin lines. Control hsp-6p::GFP shows only background GFP (i). Neuronal-specific cco-1 hairpin results in upregulation of hsp-6p::GFP in the intestine (rab-3 (ii) and unc-119 (iii) lines shown). Intestine-specific ges-1p::cco-1 hairpin (iv) also results in upregulation of the hsp-6p::GFP reporter in the intestine.

C. Fluorescent quantification of B.

FIG. 7 shows a Model for the cell non-autonomous nature of the UPR^{mt}.

Cells experiencing mitochondrial stress, in this scenario neuronal cells (circles) marked within the yellow box, produce a signal that is transmitted from the mitochondria to the nucleus to regulate the expression of genes regulated by UBL-5 and possibly DVE-1. These cells serve as sending cells and produce an extracellular signal (mitokine) that can be transmitted to distal, receiving cells, in this case intestinal cells marked in the first (“I”) box. Receiving cells perceive the mitokine and induce the mitochondrial stress response by upregulating genes regulated by UBL-5 and possibly DVE-1.

FIG. 8 shows Size comparison of cco-1 hairpin expressing worms and controls. Expression of tissuespecific hairpins does not appear to alter overall size. Four representative worms of each transgenic strain: neurons (unc-119), intestine (ges-1), and body wall muscle (myo-3) and control strains N2, sid-1, and sid-1/rol-6 are shown.

FIG. 9 shows Total average number of progeny for 30 worms of each transgenic and control strain. N2 (black) produced 290±42 progeny; sid-1(qt9) (bar second from left) produced 308±29 progeny; sid-1/rol-6 (bar third from left) produced 260±39.5 progeny; unc-119p::cco-1HP (bar third from right) produced 207±56 progeny; ges-1p::cco-1HP produced 242±39 progeny (bar second from right); myo-3p::cco-1HP (far right bar) produced 251±55 progeny.

FIG. 10 shows elk-1(e2519) lifespan is suppressed by ubl-5 RNAi. Lifespan of elk-1(e2519) weak allele on ubl-5 feeding RNAi. N2 (dark gray line) mean lifespan 18.0±0.4 days; elk-1 (e2519)

(black line) mean lifespan 20.6±0.5 days; clk-1(e2519) on ubl-5 RNAi (light gray line) mean lifespan 18.0±0.5 days. p<0.0005.

FIG. 11 shows that dve-1 RNAi shortens the lifespan of all strains tested.

A. Wildtype N2 worms fed empty vector (black line) mean lifespan 18.0±0.4 days; N2 fed dve-1 RNAi (gray line) mean 12.3±0.5 days.

B. isp-1(qm150) mutants fed empty vector (black) mean lifespan 25.8±1.0 days; isp-1-(qm150) fed dve-1 RNAi mean 12.9±0.3 days.

C. daf-1(e1370) mutants fed empty vector mean lifespan 40.7±1.2 days; daf-1(e1370) fed dve-1 RNAi mean 17.8±0.8 days.

D. eat-2(ad1116) fed empty vector mean lifespan 26.4±0.6 days; eat-2(ad1116) fed dve-1 RNAi mean 12.3±0.3 days.

FIG. 12 shows hsp-6p::GFP reporter worms at developmental stages L1 through young adult fed cco-1 RNAi from hatch. Animals were collected at the indicated stages for fluorescent microscopy. By the L3/L4 stage the UPRmt is strongly upreplated in the intestine.

FIG. 13 shows disruption of the ETC in the muscle cells which results in a shortened or wildtype lifespan can activate the hsp-6p::GFP reporter. myo-3p::cco-1HP transgenic worms were crossed to hsp-6p::GFP reporter worms show induction of the GFP reporter in the intestine.

### DETAILED DESCRIPTION

Described herein are methods of increasing the lifespan of an animal, methods of screening for compounds that increase the life span of an animal, and animals and cells that have a longer life span. More particularly, the instant invention, in certain aspects, relates to the modulation of electron transport chain function in a tissue-specific manner in an animal, for example, by modulating electron transport chain function in intestinal and/or neuronal tissue of the animal. Examples of electron transport chain components suitable for modulation in the methods and compositions of the instant invention include cytochrome c oxidase-1 subunit Vb/COX4 (cco-1) and homologs thereof. In other aspects, the instant invention relates to the modulation of the mitochondrial unfolded protein response system in an animal. Examples of mitochondrial unfolded protein response system components suitable for modulation in the methods and compositions of the instant invention include the nuclear localized ubiquitin-like protein, ubl-5 and homologs thereof. In yet other aspects, the invention provides compositions comprising one or more isolated mitokines, which, as described herein, are secreted signals from cells with reduced mitochondrial function capable of regulating the aging process. Mitokines are typically cell non-autonomous signals produced in a cell of an animal with reduced mitochondrial function.

Mitochondria display extensive morphological differences across tissues and during different development stages, varying in size, shape, and biochemical activity depending upon which tissue they are derived (Copeland et al., 2009; reviewed in Kuznetsov et al., 2009). Tissues in which the energetic demand is high, such as muscle and neurons, have high numbers of mitochondria per cell (Kwong and Sohal, 2000; Rossignol et al., 2000; Tsang et al., 2001). Thus, one might expect that a loss of mitochondrial function in one tissue might affect aging to a greater extent than loss in a different tissue, even in the absence of secreted signaling between the tissues. A priori it would seem any mutation or perturbation of the mitochondrial ETC would result in sickness and early death, especially in complex animals such as mammals; thus, it is surprising that loss of ETC function increases the life span of worms, flies and mice. Although multiple distinct mutations and RNAi knockdown of nuclear encoded ETC components result in increased longevity, it appears that the level to which these individual genes are reduced is key to the extended lifespan observed. For example, strong RNAi knockdown of atp-3, a subunit of the ATP synthase, leads to early larval arrest or lethality in the worm. However, weaker knockdown of the same mitochondrial gene almost doubles the animal's lifespan (Rea et al., 2007). Similarly, Surf1 mutant mice have 30-50% reduced COX activity and increased longevity, yet Leigh patients carrying Surf1 mutations have almost 70% reduced COX activity resulting in death during childhood or early adulthood (Dell'Agnello et al., 2007). Taken together, there appears to be a conserved mitochondrial longevity pathway only revealed at optimal levels of mitochondrial gene reduction.

One possible suggestion for the observation of increased longevity under conditions of reduced mitochondrial function has come from the “rate of living” theory of aging, in existence for almost a hundred years, which suggest that the metabolic expenditures of an organism ultimately determine its life span (Pearl, 1928; Rubner, 1908). A modification to this theory has suggested alternatively that, because reactive oxygen species (ROS) are generated as a byproduct of the metabolic activity of the mitochondrial ETC during the production of ATP (Harman, 1956), a decrease in ROS production is the major contributing factor to the long-lived phenotypes of ETC mutants (Feng et al., 2001; Rea and Johnson, 2003). Recent evidence has questioned this assumption, (Yang et al., 2007; Vanraamsdonk et al., 2009; Copeland, 2009; reviewed Gems 2009), and does not support a linear relationship between ROS production and life span. With the increased skepticism towards the oxidative stress theory of aging comes the question: if not by manipulation of ROS in a cell-autonomous manner, then by what mechanism does reduction of mitochondrial function affect aging?

Applicants attempted to address this question by asking whether manipulations to ETC function could regulate aging in a cell non-autonomous fashion in the nematode C. elegans. Applicants addressed whether key tissues could govern increases in longevity when components of the mitochondrial ETC are inactivated. Applicants reasoned that if they could identify the crucial tissues from which the ETC longevity pathway functions, they could begin to identify the origin of the longevity signal and perhaps potential mediators of this signal.

Applicants have discovered that not only are specific tissues required for the ETC response to aging, they can initiate a protective response in other tissues and organs within the animal. As detailed herein, evidence is disclosed of a secreted signal from cells with reduced mitochondrial function capable of regulating the aging process, genes important and specific for this response and a novel implication for the mitochondrial unfolded protein response in the execution of this signal.

Applicants have identified key tissues, genes important and specific for mitochondrial longevity and at least one mechanism that is necessary for increased longevity in response to altered mitochondrial function in a metazoan. Using either a dsRNA hairpin approach or the rde-1 complementation system, Applicants found that reduction of cco-1 in the intestine or the nervous system resulted in increased longevity. Applicants' discovery suggests that there exists a signaling system that can originate in the mitochondria in either the neurons or the intestine that is transmitted throughout the organism to determine, and possibly coordinate, the rate of aging among all tissues. Much like the discovery of cco-1 RNAi induced lifespan extension, the increased lifespan due to tissue specific knockdown of cco-1 was daf-16 independent. cco-1 RNAi results in reduced size, growth rates, movement and fertility. Applicants found that the long-lived intestinal or neuronal cco-1 RNAi animals did not suffer from these adversities. Therefore, it appears that within an organism, tissue specific modulation of the ETC can affect longevity without many of the detrimental side effects of global reduction of the ETC within the entire organism. Consistent with this idea, Applicants found that knockdown in the muscle cells resulted in slowed movement, but did not result in increased longevity. Reduction of the ETC by RNAi also affects many other stress responses including UV, oxidative, and heat stress (Lee et al., 2002; Copeland et al., 2009; Kuznetsov et al., 2009). However, Applicants found that these responses were not central to the increased longevity of the intestinal or neuronal ETC RNAi knockdown. Instead, the mitochondrial unfolded protein response, UPRmt, was important for the extended longevity of ETC mutant animals and has been previously reported to be upregulated in response to RNAi of cco-1 (Yoneda et al., 2004). Consistent with the temporal requirements of the ETC to modulate longevity during the L3/L4 larval stages, Applicants found that UPRmt could only be induced when cco-1 RNA; was administered before the L3/L4 larval stage, but not in adulthood, consistent with the temporal requirements of cco-1 for longevity. Therefore, induction of the UPRmt mirrored the temporal requirements of the ETC to promote longevity when reduced. More importantly, the fact that induction of the UPRmt can be maintained long into adulthood, well after the mitochondrial insult had been given in larval development, indicates that the animal might possess an epigenetic mechanism to ensure increased resistance to future mitochondrial perturbations.

Of the currently identified UPRmt pathway members, Applicants found that the ubiquitin like protein, UBL-5, which provides transcriptional specificity for the homeobox transcription factor DVE-1 in response to unfolded proteins in the mitochondria, is essential for the increased longevity of ETC mutant animals. Interestingly, ubl-5 is specific for mitochondrial ETC RNAi longevity since knockdown of ubl-5 did not affect the lifespan of wild type animals or long-lived animals with reduced insulin/IGF-1 signaling or diet restricted animals. Consistent with the UPRmt being important and specific for ETC mediated longevity, Applicants found that the UPRmt is not induced by either the IIS or diet restriction longevity pathways, but is induced by cco-1 RNAi (Haynes et al., 2007). Both dve-1 and clpp-1 reduction appeared to make animals sick as RNAi reduction reduced lifespan of wild type animals and all long-lived mutant animals tested. Therefore, much like the specificity inscribed for daf-16 upon the insulin/IGF-1 signaling pathways and pha-4 upon the diet restriction pathway, ubl-5 appears to provide specificity for the ETC longevity pathway.

While ubl-5 is important and specific for the increased longevity of animals with reduced mitochondrial function, it is likely that overexpression of ubl-5 will not be sufficient for increased longevity, nor will ectopic induction of the UPRmt as a few lines of evidence suggests. First, Applicants found that muscle specific cco-1 RNAi could also induce the intestinal hsp-6p::GFP reporter, yet these animals were not long lived (FIG. S6). Second, Applicants find that short-lived mev-1 mutant animals also induce the UPRmt. Third, many of the nuclear encoded mitochondrial genes discovered to induce the UPRmt when inactivated using RNAi (Yoneda et al., 2004) are not long lived. Therefore, while many of these perturbations have pleiotropic effects that result in their short lifespan, their ability to upregulate the UPRmt is not sufficient to overcome these potentially harmful side effects.

One of the most surprising findings by Applicants is the UPRmt can be activated in a cell non-autonomous manner. Because the hsp-6p::GFP reporter is primarily limited to expression in the intestine, Applicants asked if perturbation of cco-1 in the nervous system could induce the UPRmt in the intestine. The nervous system does not innervate the intestine of the worm. Therefore, finding that neuronal limited knockdown of cco-1 could profoundly induce the hsp-6 reporter indicates that a cue from the nervous system must travel to the intestine to induce the UPRmt (FIG. 7). It is clear that production of this cue in a limited number of cells can profoundly influence the survival of the entire organism. Because this signal is the product of perceived mitochondrial stress that results in increased survival, Applicants have termed this cell non-autonomous signal a “mitokine.” In certain embodiments, a mitokine is proteinacious. In other embodiments, a mitokine is a small molecule.

It is intriguing to speculate why reduced mitochondrial ETC in only a few tissues are able to send a pro-longevity cue, or mitokine, but others do not. Because the intestine and the sensory neurons (amphids and phasmids) are the only cells that are in direct contact with the environment (the hypodermis/skin is wrapped in a protective, dense cuticle), perhaps these cells are fine-tuned to perceive mitochondrial insults that might be present in the worm's environment (soil). Many mitochondrial toxins are produced by bacteria present in the soil, the food source of C. elegans, such as some of the most common toxins including antimycin A, oligomycin, and valinomycin produced by many different Streptomyces species. Perhaps perception of these mitochondrial toxins in the nervous system and intestine can create a rudimentary defense mechanism to protect naive cells to incoming insults by upregulating the UPRmt in a systemic manner. Intriguingly, low doses of Antimycin A results in increased longevity of C. elegans (Dillin 2002). Alternatively, mitochondrial metabolism in the nervous system and intestine might have different requirements than other tissues making disruptions in these tissues more susceptible to perturbation and subsequent UPRmt upregulation. There is a growing body of research emphasizing the importance of ROS, not as damaging agents, but as crucial components of cell signaling. It remains a possibility that ROS may act as signaling molecules and potentially serve as the mitokine or intermediary to elicit a nuclear response.

Mitochondria have been important in longevity research in yeast, worms, flies and rodents to connect rates of metabolism with longevity. The initial finding that reduction of mitochondrial function during a specific time in the worm's life cycle that could be uncoupled from mitochondrial ETC metabolic activity suggested that mitochondria might establish the rate of aging in a manner that is independent of previously anticipated modes, such as generation of reactive oxygen species. Consistent with this, data in worms and flies has shown that resistance to oxidative damage does not correlate with several long-lived mitochondrial reduced animals (Copeland et al., 2009; Kuznetsov et al., 2009; Lee et al., 2002).

As described herein, Applicants have identified the nervous system and intestinal cells as being key mediators of a signaling pathway that requires the UPRmt. Applicants' discovery of the mitokine can provide a novel avenue to explore treatment of mitochondrial diseases in a tissue and cell type specific manner if conserved from worm to man.

As used herein, the term “electron transport chain component” or “ETC component” refers to any constituent involved in the regulation and/or activity of an electron transport chain in an organism. For example, ETC components include any of the constituents that comprise Complex I, Complex II, Complex III, Complex IV, or Complex V (ATP synthase) of the electron transport chain in a mitochondrion of a eukaryote. ETC components encompass, without limitation, any protein, protein subunit, peptide chain and fractions thereof as well as any prosthetic groups (e.g., FMN, heme groups, iron-sulfur structures) or other constituent, such as, e.g., ubiquinone, involved in the regulation and/or activity of an electron transport chain in an organism. ETC components also encompass any nucleic acids coding for a protein, protein subunit, peptide chain or fraction thereof involved in the regulation and/or activity of an electron transport chain in an organism.

The term “mitochondrial unfolded protein response component” refers to any constituent involved in the regulation and/or activity of the mitochondrial unfolded protein response in an organism. Mitochondrial unfolded protein response components also encompass any nucleic acids coding for a protein, protein subunit, peptide chain or fraction thereof involved in the regulation and/or activity of a mitochondrial unfolded protein response.

As used herein, the terms “polynucleotides” and “nucleic acid sequences” refer to DNA, RNA and cDNA sequences and include all analogs and backbone substitutes such as PNA that one of skill in the art would recognize as capable of substituting for naturally occurring nucleotides and backbones thereof.

“Antisense” nucleic acids are DNA or RNA molecules that are complementary to at least a portion of a specific mRNA molecule (Weintraub, Scientific American 262 40, 1990). In the cell, the antisense nucleic acids hybridize to the corresponding mRNA, forming a double-stranded molecule. This interferes with the translation of the mRNA since the cell will not translate an mRNA that is double-stranded. Antisense oligomers of at least about 15, about 20, about 25, about 30, about 35, about 40, or of at least about 50 nucleotides are preferred, since they are easily synthesized and are less likely to cause non-specific interference with translation than larger molecules. The use of antisense methods to inhibit the in vitro translation of genes is well known in the art (Marcus-Sakura Anal. Biochem. 172: 289, 1998). In the present case, animals transformed with constructs containing antisense fragments of an electron transport chain or mitochondrial unfolded protein response gene, such as for example, cco-1 or ubl-5 in Caenorhabditis elegans and homologs thereof, would display a modulated phenotype such as altered longevity.

The invention provides for nucleic acids complementary to (e.g., antisense sequences to) cellular modulators of ETC or UPRmt activity. Antisense sequences are capable of inhibiting the transport, splicing or transcription of protein-encoding genes, e.g., nucleic acids encoding cco-1 or ubl-5 in Caenorhabditis elegans. The inhibition can be effected through the targeting of genomic DNA or messenger RNA. The transcription or function of targeted nucleic acid can be inhibited, for example, by hybridization and/or cleavage. One particularly useful set of inhibitors provided by the present invention includes oligonucleotides which are able to either bind gene or message, in either case preventing or inhibiting the production or function of the protein. The association can be through sequence specific hybridization. Another useful class of inhibitors includes oligonucleotides that cause inactivation or cleavage of protein message. The oligonucleotide can have enzyme activity which causes such cleavage, such as ribozymes. The oligonucleotide can be chemically modified or conjugated to an enzyme or composition capable of cleaving the complementary nucleic acid. One can screen a pool of many different such oligonucleotides for those with the desired activity.

Short double-stranded RNAs (dsRNAs; typically <30 nucleotides) can be used to silence the expression of target genes in animals and animal cells. Upon introduction, the long dsRNAs enter the RNA interference (RNAi) pathway which involves the production of shorter (20-25 nucleotide) small interfering RNAs (siRNAs) and assembly of the siRNAs into RNA-induced silencing complexes (RISCs). The siRNA strands are then unwound to form activated RISCs, which cleave the target RNA. Double stranded RNA has been shown to be extremely effective in silencing a target RNA. Introduction of double stranded RNA corresponding to an ETC component gene or UPRmt gene would be expected to modify the ETC and UPRmt-related functions discussed herein including, but not limited to, longevity.

General methods of using antisense, ribozyme technology and RNAi technology, to control gene expression, or of gene therapy methods for expression of an exogenous gene in this manner are well known in the art. Each of these methods utilizes a system, such as a vector, encoding either an antisense or ribozyme transcript. The term “RNAi” stands for RNA interference. This term is understood in the art to encompass technology using RNA molecules that can silence genes. See, for example, McManus, et al. Nature Reviews Genetics 3: 737, 2002. In this application, the term “RNAi” encompasses molecules such as short interfering RNA (siRNA), small hairpin or short hairpin RNA (shRNA), microRNAs, and small temporal RNA (stRNA). Generally speaking, RNA interference results from the interaction of double-stranded RNA with genes.

“Small interfering RNA” (siRNA) refers to double-stranded RNA molecules from about 10 to about 30 nucleotides long that are named for their ability to specifically interfere with protein expression through RNA interference (RNAi). Preferably, siRNA molecules are 12-28 nucleotides long, more preferably 15-25 nucleotides long, still more preferably 19-23 nucleotides long and most preferably 21-23 nucleotides long. Therefore, preferred siRNA molecules are 12, 13, 14, 15, 16, 17, 18, 19, 20, 21, 22, 23, 24, 25, 26, 27 28 or 29 nucleotides in length.

RNAi is a two-step mechanism. Elbashir et al., Genes Dev., 15: 188-200, 2001. First, long dsRNAs are cleaved by an enzyme known as Dicer in 21-23 ribonucleotide (nt) fragments, called small interfering RNAs (siRNAs). Then, siRNAs associate with a ribonuclease complex (termed RISC for RNA Induced Silencing Complex) which target this complex to complementary mRNAs. RISC then cleaves the targeted mRNAs opposite the complementary siRNA, which makes the mRNA susceptible to other RNA degradation pathways.

siRNAs of the present invention are designed to interact with a target ribonucleotide sequence, meaning they complement a target sequence sufficiently to bind to the target sequence. The present invention also includes siRNA molecules that have been chemically modified to confer increased stability against nuclease degradation, but retain the ability to bind to target nucleic acids that may be present.

The invention provides antisense oligonucleotides capable of binding messenger RNA, e.g., mRNA encoding cco-1 or ubl-5 in C. elegans, that can inhibit polypeptide activity by targeting mRNA. Strategies for designing antisense oligonucleotides are well described in the scientific and patent literature, and the ordinarily skilled artisan can design such oligonucleotides using the novel reagents of the invention. For example, gene walking/RNA mapping protocols to screen for effective antisense oligonucleotides are well known in the art, see, e.g., Ho, Methods Enzymol. 314: 168-183, 2000, describing an RNA mapping assay, which is based on standard molecular techniques to provide an easy and reliable method for potent antisense sequence selection. See also Smith, Eur. J. Pharm. Sci. 11: 191-198, 2000.

Naturally occurring nucleic acids are typically used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can also be used. For example, peptide nucleic acids (PNAs) containing non-ionic backbones, such as N-(2-aminoethyl)glycine units can be used. Antisense oligonucleotides having phosphorothioate linkages can also be used, as described in Mata, Toxicol Appl Pharmacol. 144: 189-197, 1997; Antisense Therapeutics, ed. Agrawal, Humana Press, Totowa, N.J., 1996. Antisense oligonucleotides having synthetic DNA backbone analogues can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids, as described above.

Combinatorial chemistry methodology can be used to create vast numbers of oligonucleotides that can be rapidly screened for specific oligonucleotides that have appropriate binding affinities and specificities toward any target, such as the sense and antisense polypeptides sequences of the invention (see, e.g., Gold, J. of Biol. Chem. 270: 13581-13584, 1995).

As used herein, the term “homolog” covers identity with respect to structure and/or function providing the expression product of the resultant nucleotide sequence has the inhibitory or upregulatory activity. With respect to sequence identity, preferably there is at least 70%, more preferably at least 80%, more preferably at least 90% sequence identity. More preferably there is at least 95%, more preferably at least 98%, sequence identity. These terms also encompass allelic variations of the sequences. The term, homolog, may apply to the relationship between genes separated by the event of speciation or to the relationship between genes separated by the event of genetic duplication.

Relative sequence identity can be determined by commercially available computer programs that can calculate % identity between two or more sequences using any suitable algorithm for determining identity, using for example default parameters. A typical example of such a computer program is CLUSTAL. Advantageously, the BLAST algorithm is employed, with parameters set to default values. The BLAST algorithm is described in detail at http://www.ncbi.nih.gov/BLAST/blast_help.html, which is incorporated herein by reference.

The homologs of the peptides as provided herein typically have structural similarity with such peptides. A homolog of a polypeptide includes one or more conservative amino acid substitutions, which may be selected from the same or different members of the class to which the amino acid belongs. By way of non-limiting example, an amino acid belonging to a grouping of amino acids having a particular size or characteristic (such as charge, hydrophobicity and hydrophilicity) may generally be substituted for another amino acid without substantially altering the structure of a polypeptide.

Examples of homologs according to the invention include cco-1 homologs, such as nucleotides with at least 70%, more preferably at least 80%, more preferably at least 90%, more preferably at least 95%, more preferably at least 98% sequence identity to the nucleotide sequence depicted in GenBank Accession No. NM_{—}060200. Examples of homologs according to the invention also include ubl-5 homologs, such as nucleotides with at least 70%, more preferably at least 80%, more preferably at least 90%, more preferably at least 95%, more preferably at least 98% sequence identity to the nucleotide sequence depicted in GenBank Accession No. NM_{—}059239.

In yet other embodiments, homologs according to the invention include cco-1 homologs, such as peptides with at least 70%, more preferably at least 80%, more preferably at least 90%, more preferably at least 95%, more preferably at least 98% sequence identity to the amino acid sequence depicted in GenBank Accession No. NP_{—}492601. Examples of homologs according to the invention also include ubl-5 homologs, such as peptides with at least 70%, more preferably at least 80%, more preferably at least 90%, more preferably at least 95%, more preferably at least 98% sequence identity to the amino acid sequence depicted in GenBank Accession No. NP_{—}491640.

Another embodiment of the invention relates to animals that have at least one modulated ETC or UPRmt function. Such modulated functions include among others an altered longevity. “Longevity” refers to the life span of an animal. Thus, longevity refers to the number of years in the life span of an animal. “Stress tolerance” refers to an animal's ability to tolerate exposure to various internal and external environmental challenges such as exposure to UV light, exposure to high osmolarity, exposure to infection, exposure to oxidative damage, exposure to metal compounds, and exposure to certain toxins. Those of skill in the art will recognize that an increase in the lifespan of an animal can readily be measured by various assays known in the art. The field of gerontology is one such example of a relevant art. By way of example, longevity may be assessed by various markers such as number of generations to senescence in non-immortalized somatic cells, graying hair, wrinkling, and other such alterations in physiological markers associated with aging. Those of ordinary skill in the art will also recognize that alterations in an animal's ability to tolerate stress, i.e., its response to stress, may be assessed by various assays, including by way of example, by assessing changes in expression or activity of molecules involved in the stress response by measuring expression of stress response genes, protein levels of specific stress response proteins, or activity levels of specific stress response proteins.

Animals having a modified ETC or UPRmt-related function include transgenic animals with an altered longevity due to transformation with constructs using antisense or siRNA technology that affect transcription or expression from an ETC or UPRmt component gene. Such animals exhibit an altered longevity.

Accordingly, in another series of embodiments, the present invention provides methods of screening or identifying proteins, small molecules or other compounds which are capable of inducing or inhibiting the activity or expression of ETC and/or UPRmt component genes and proteins. The assays may be performed, by way of example, in vitro using transformed or non-transformed cells, immortalized cell lines, or in vivo using transformed animal models enabled herein.

In another series of embodiments, the present invention provides methods for identifying proteins and other compounds which bind to, or otherwise directly interact with an ETC and/or UPRmt component protein. Thus, in one series of embodiments, High Throughput Screening-derived proteins, DNA chip arrays, cell lysates or tissue homogenates may be screened for proteins or other compounds which bind to one of the normal or mutant ETC or UPRmt genes. Alternatively, any of a variety of exogenous compounds, both naturally occurring and/or synthetic (e.g., libraries of small molecules or peptides), may be screened for ETC and/or UPRmt function modulating capacity.

Embodiments of the invention also include methods of identifying proteins, small molecules and other compounds capable of modulating the activity of the ETC and/or UPRmt. Using normal cells or animals, the transformed cells and animal models of the present invention, or cells obtained from subjects bearing normal or mutant ETC and/or UPRmt component genes, the present invention provides methods of identifying such compounds on the basis of their ability to affect the expression of an ETC and/or UPRmt component, the activity of an ETC and/or UPRmt component, the activity of proteins that interact with normal or mutant ETC and/or UPRmt component proteins, or other biochemical, histological, or physiological markers that distinguish cells bearing normal and modulated ETC and/or UPRmt activity in animals.

In accordance with another aspect of the invention, the proteins of the invention can be used as starting points for rational chemical design to provide ligands or other types of small chemical molecules. Alternatively, small molecules or other compounds identified by the above-described screening assays may serve as “lead compounds” in design of modulators of ETC and/or UPRmt-related traits, such as longevity, in animals.

DNA sequences encoding an ETC and/or UPRmt component protein can be expressed in vitro by DNA transfer into a suitable host cell. “Host cells” are cells in which a vector can be propagated and its DNA expressed. The term also includes any progeny or graft material, for example, of the subject host cell. It is understood that all progeny may not be identical to the parental cell since there may be mutations that occur during replication. However, such progeny are included when the term “host cell” is used. Methods of stable transfer, meaning that the foreign DNA is continuously maintained in the host, are known in the art.

The terms “recombinant expression vector” or “expression vector” refer to a plasmid, virus or other vehicle known in the art that has been manipulated by insertion or incorporation of a genetic sequence. Such expression vectors contain a promoter sequence which facilitates the efficient transcription of the inserted sequence. The expression vector typically contains an origin of replication, a promoter, as well as specific genes that allow phenotypic selection of the transformed cells.

Methods that are well known to those skilled in the art can be used to construct expression vectors containing an ETC and/or UPRmt component coding sequence and appropriate transcriptional/translational control signals. These methods include in vitro recombinant DNA techniques, synthetic techniques, and in vivo recombination/genetic techniques.

A variety of host-expression vector systems may be utilized to express a coding sequence. These include but are not limited to microorganisms such as bacteria transformed with recombinant bacteriophage DNA, plasmid DNA or cosmid DNA expression vectors containing a coding sequence; yeast transformed with recombinant yeast expression vectors containing a coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing a coding sequence; insect cell systems infected with recombinant virus expression vectors (e.g., baculovirus) containing a coding sequence; or animal cell systems infected with recombinant virus expression vectors (e.g., retroviruses, adenovirus, vaccinia virus) containing a coding sequence, or transformed animal cell systems engineered for stable expression.

Depending on the host/vector system utilized, any of a number of suitable transcription and translation elements, including constitutive and inducible promoters, transcription enhancer elements, transcription terminators, etc. may be used in the expression vector (see e.g., Bitter et al. Methods in Enzymology 153, 516-544, 1987). For example, when cloning in bacterial systems, inducible promoters such as pL of bacteriophage 7, plac, ptrp, ptac (ptrp-lac hybrid promoter) and the like may be used. When cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter) may be used.

The term “operably linked” refers to functional linkage between a promoter sequence and a nucleic acid sequence regulated by the promoter. The operably linked promoter controls the expression of the nucleic acid sequence.

The expression of structural genes may be driven by a number of promoters. Although the endogenous, or native promoter of a structural gene of interest may be utilized for transcriptional regulation of the gene, preferably, the promoter is a foreign regulatory sequence. For mammalian expression vectors, promoters capable of directing expression of the nucleic acid preferentially in a particular cell type may be used (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

Promoters useful in the invention include both natural constitutive and inducible promoters as well as engineered promoters. Examples of inducible promoters useful in animals include those induced by chemical means, such as the yeast metallothionein promoter, which is activated by copper ions (Mett, et al. Proc. Natl. Acad. Sci., U.S.A. 90, 4567, 1993); and the GRE regulatory sequences which are induced by glucocorticoids (Schena, et al. Proc. Natl. Acad. Sci., U.S.A. 88, 10421, 1991). Other promoters, both constitutive and inducible will be known to those of ordinary skill in the art.

Animals included in the invention are any animals amenable to transformation techniques, including vertebrate and non-vertebrate animals and mammals. Examples of mammals include, but are not limited to, pigs, cows, sheep, horses, cats, dogs, chickens, or turkeys.

Compounds tested as modulators of ETC and/or UPRmt activity can be any small organic molecule, or a biological entity, such as a protein, e.g., an antibody or peptide, a sugar, a nucleic acid, e.g., an antisense oligonucleotide, RNAi, or a ribozyme, or a lipid. Alternatively, modulators can be genetically altered versions of a cellular modulator of ETC and/or UPRmt activity. Typically, test compounds will be small organic molecules, nucleic acids, peptides, lipids, and lipid analogs.

Essentially any chemical compound can be used as a potential modulator or ligand in the assays of the invention, although most often compounds can be dissolved in aqueous or organic solutions. In certain embodiments, the assays of the invention are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one embodiment, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37: 487-493, 1991 and Houghton et al., Nature 354: 84-88, 1991). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90: 6909-6913, 1993), vinylogous polypeptides (Hagihara et al., J. Amer. Chem. Soc. 114: 6568, 1992), nonpeptidal peptidomimetics with glucose scaffolding (Hirschmann et al., J. Amer. Chem. Soc. 114: 9217-9218, 1992), analogous organic syntheses of small compound libraries (Chen et al., J. Amer. Chem. Soc. 116: 2661, 1994), oligocarbamates (Cho et al., Science 261: 1303, 1993), and/or peptidyl phosphonates (Campbell et al., J. Org. Chem. 59: 658, 1994), nucleic acid libraries (see Ausubel, Berger and Sambrook, all supra), peptide nucleic acid libraries (see, e.g., U.S. Pat. No. 5,539,083), antibody libraries (see, e.g., Vaughn et al, Nature Biotechnology, 14: 309-314, 1996 and PCT/US96/10287), carbohydrate libraries (see, e.g., Liang et al., Science 274: 1520-1522, 1996 and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, R U, 3D Pharmaceuticals, Exton, Pa., Martek Biosciences, Columbia, Md., etc.).

Candidate compounds are useful as part of a strategy to identify drugs for enhancing longevity wherein the compounds modulate activity of cellular molecules regulated by the ETC and/or UPRmt, for example, wherein the compound modulates the activity of cco-1, ubl-5, or a homolog thereof. Screening assays for identifying candidate or test compounds that bind to one or more cellular modulators of ETC and/or UPRmt activity, or polypeptides or biologically active portions thereof, are also included in the invention. The test compounds can be obtained using any of the numerous approaches in combinatorial library methods known in the art, including, but not limited to, biological libraries; spatially addressable parallel solid phase or solution phase libraries; synthetic library methods requiring deconvolution; the “one-bead one-compound” library method; and synthetic library methods using affinity chromatography selection. The biological library approach can be used for, e.g., peptide libraries, while the other four approaches are applicable to peptide, non-peptide oligomer or small molecule libraries of compounds (Lam, Anticancer Drug Des. 12: 145, 1997). Examples of methods for the synthesis of molecular libraries can be found in the art, for example in: DeWitt et al., Proc. Natl. Acad. Sci. U.S.A. 90: 6909, 1993; Erb et al., Proc. Natl. Acad. Sci. USA 91: 11422, 1994; Zuckermann et al., J. Med. Chem. 37: 2678, 1994; Cho et al., Science 261: 1303, 1993; Carrell et al., Angew. Chem. Int. Ed. Engl. 33: 2059, 1994; Carell et al., Angew. Chem. Int. Ed. Engl. 33: 2061, 1994; and Gallop et al., J. Med. Chem. 37: 1233, 1994.

Libraries of compounds can be presented in solution (e.g., Houghten, Bio/Techniques 13: 412-421, 1992), or on beads (Lam, Nature 354: 82-84, 1991), chips (Fodor, Nature 364: 555-556, 1993), bacteria (U.S. Pat. No. 5,223,409), spores (U.S. Pat. Nos. 5,571,698, 5,403,484, and 5,223,409), plasmids (Cull et al., Proc. Natl. Acad. Sci. USA 89: 1865-1869, 1992) or on phage (Scott et al., Science 249: 386-390, 1990; Devlin, Science 249: 404-406, 1990; Cwirla et al., Proc. Natl. Acad. Sci. USA 87: 6378-6382, 1990; and Felici, J. Mol. Biol. 222: 301-310, 1991).

This invention further pertains to novel agents identified by the herein-described screening assays and uses thereof for treatments as described herein, for example, for the enhancement of longevity in an animal, including humans.

In one embodiment the invention provides soluble assays using a cellular modulator of ETC and/or UPRmt activity, or a cell or tissue expressing a cellular modulator of ETC and/or UPRmt activity, either naturally occurring or recombinant. In another embodiment, the invention provides solid phase based in vitro assays in a high throughput format, where a cellular modulator of ETC and/or UPRmt activity is attached to a solid phase substrate via covalent or non-covalent interactions.

“Inhibitors,” “activators,” and “modulators” of ETC and/or UPRmt activity in cells are used to refer to inhibitory, activating, or modulating molecules, respectively, identified using in vitro and in vivo assays for ETC and/or UPRmt activity, e.g., ligands, agonists, antagonists, and their homologs and mimetics.

“Modulator” includes inhibitors and activators. Inhibitors are agents that, e.g., bind to, partially or totally block stimulation, decrease, prevent, delay activation, inactivate, desensitize, or down regulate ETC and/or UPRmt activity, e.g., antagonists. Activators are agents that, e.g., bind to, stimulate, increase, open, activate, facilitate, enhance activation, sensitize or up regulate ETC and/or UPRmt activity, e.g., agonists. Modulators include genetically modified versions of biological molecules with ETC and/or UPRmt activity, e.g., with altered activity, as well as naturally occurring and synthetic ligands, antagonists, agonists, small chemical molecules and the like.

“Cell-based assays” for inhibitors and activators include, e.g., applying putative modulator compounds to a biological sample having ETC and/or UPRmt activity and then determining the functional effects on ETC and/or UPRmt activity, as described herein. “Cell based assays” include, but are not limited to, in vivo tissue or cell samples from a mammalian subject or in vitro cell-based assays comprising a biological sample having ETC and/or UPRmt activity that are treated with a potential activator, inhibitor, or modulator and are compared to control samples without the inhibitor, activator, or modulator to examine the extent of inhibition.

“Compound” or “test compound” refers to any compound tested as a modulator of ETC and/or UPRmt activity. The test compound can be any small organic molecule, or a biological entity, such as a protein, e.g., an antibody or peptide, a sugar, a nucleic acid, e.g., an antisense oligonucleotide, RNAi, or a ribozyme, or a lipid. Alternatively, a test compound can be modulators of biological activities that affect an ETC and/or UPRmt activity. Typically, test compounds will be small organic molecules, nucleic acids, peptides, lipids, or lipid analogs.

GenBank Accession No. NM_{—}060200 is an example of a cco-1 nucleotide sequence. GenBank Accession No. NM_{—}059239 is an example of a ubl-5 nucleotide sequence.

The invention will now be further described by way of the following non-limiting examples.

### Example 1

### cco-1 Functions in Specific Tissues to Affect the Aging Process

The aim of this example is to show that tissue-specific ETC knockdown can alter the lifespan of an organism by demonstrating that cco-1 functions in specific tissues to affect the aging process.

Because mitochondria are tailored to meet tissue-specific requirements and the ETC is part of a longevity cue during a specific period in the animal's life cycle, the L3/L4 larval stage, Applicants hypothesized that there might be a tissue-specific component of the ETC mediated longevity pathway. To ascertain whether tissue-specific ETC knockdown could alter the lifespan of an organism, Applicants created transgenic worm lines carrying an inverted repeat hairpin (HP) directed towards the nuclear encoded cytochrome c oxidase-1 subunit Vb/COX4 (cco-1). cco-1 was chosen because knockdown of this gene results in intermediate phenotypes compared to knockdown of the other ETC genes by RNAi, allowing both positive and negative modulation of longevity to be identified (Dillin et al., 2002b; Lee et al., 2002; Rea et al., 2007). Furthermore, cco-1 RNAi does not result in detrimental phenotypes observed when bacterial feeding RNAi against other components of the ETC is administered undiluted, such as severe developmental delay and lethality (Rea et al., 2007).

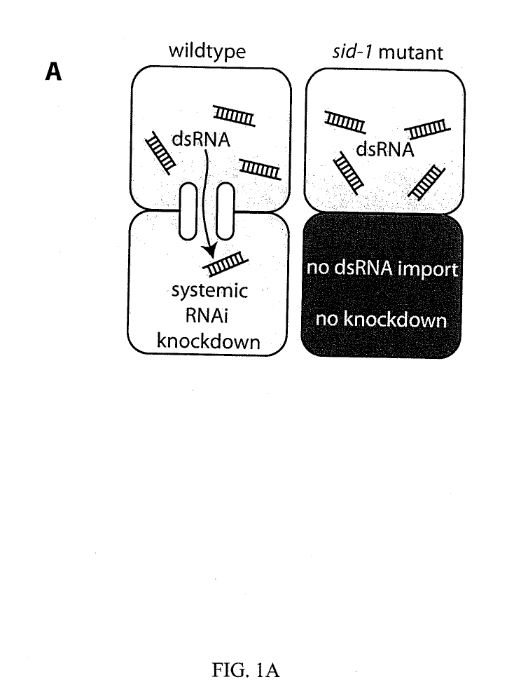

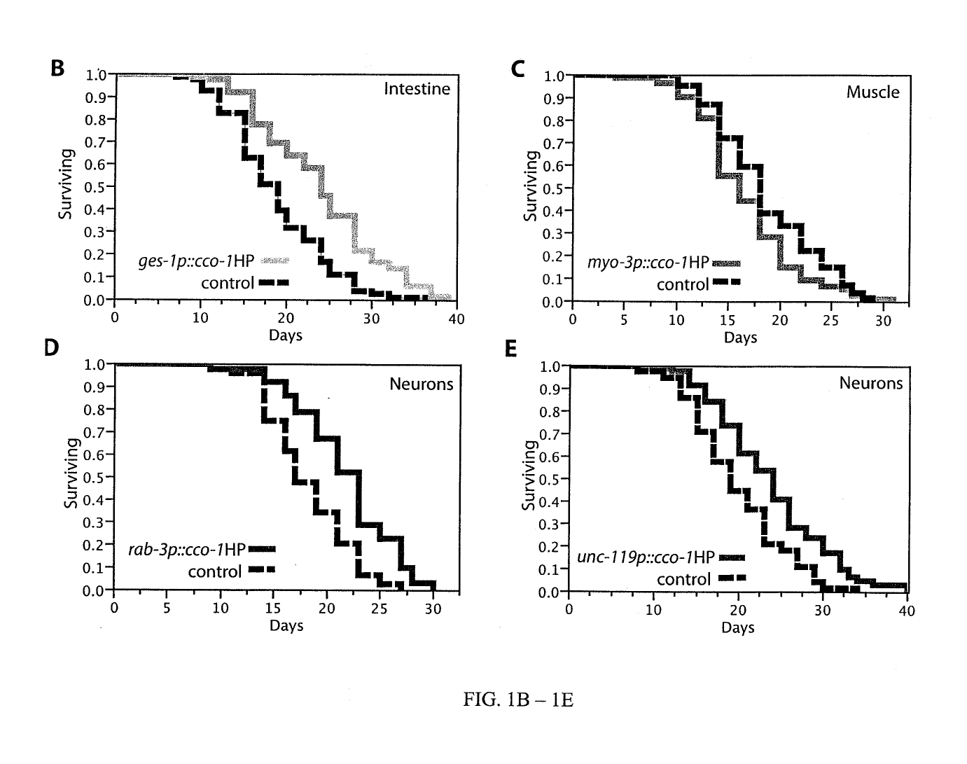

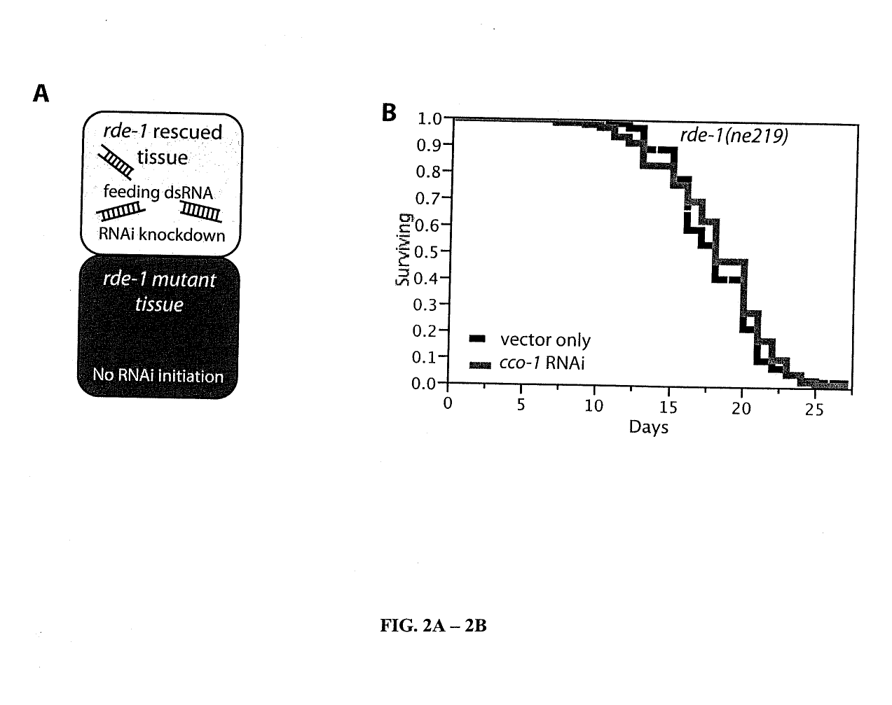

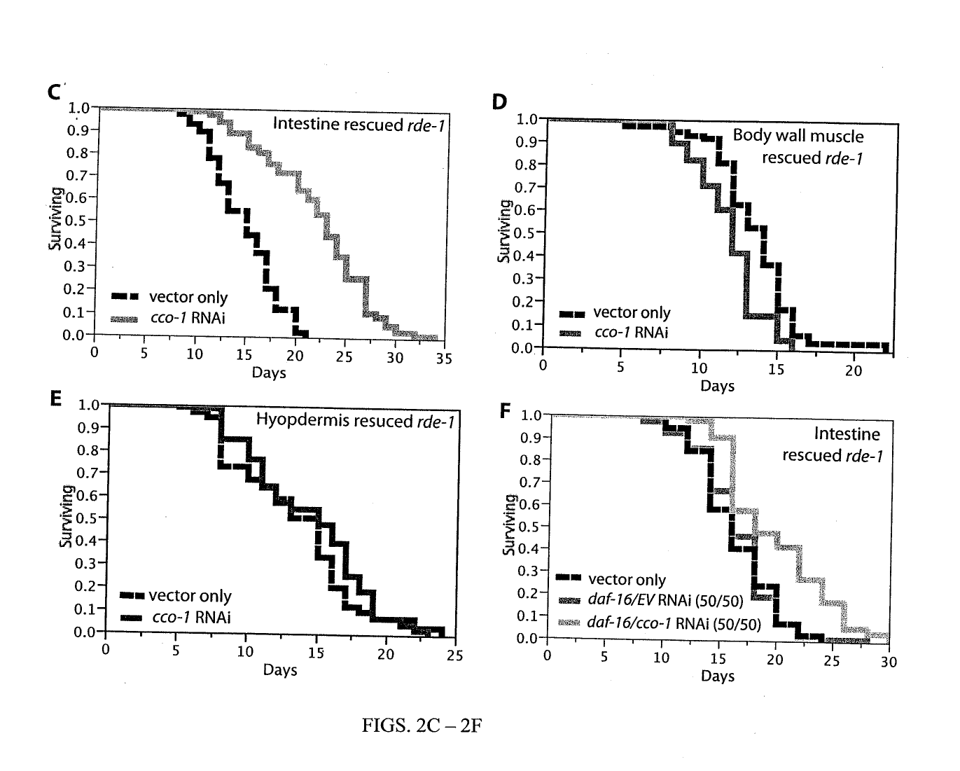

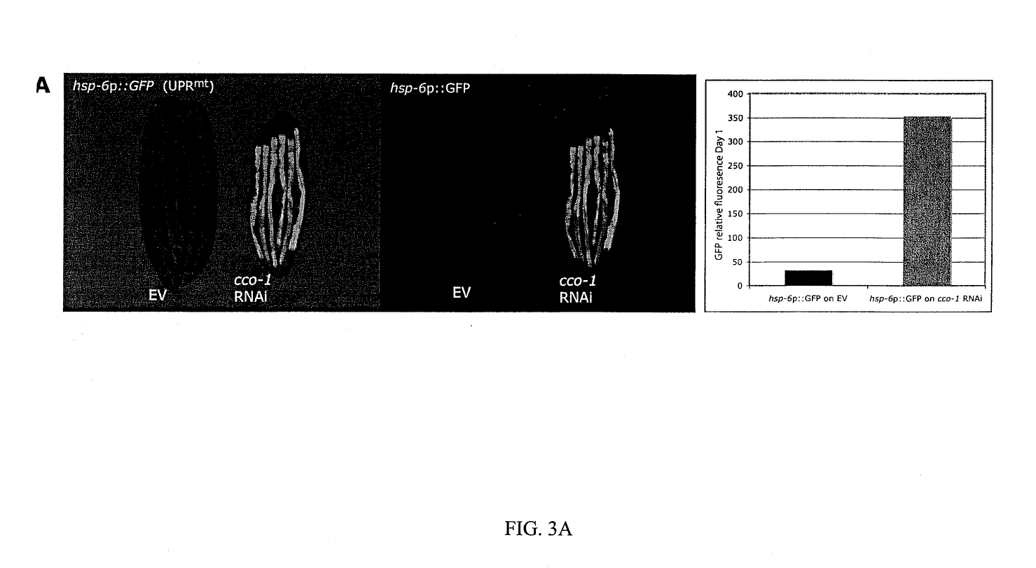

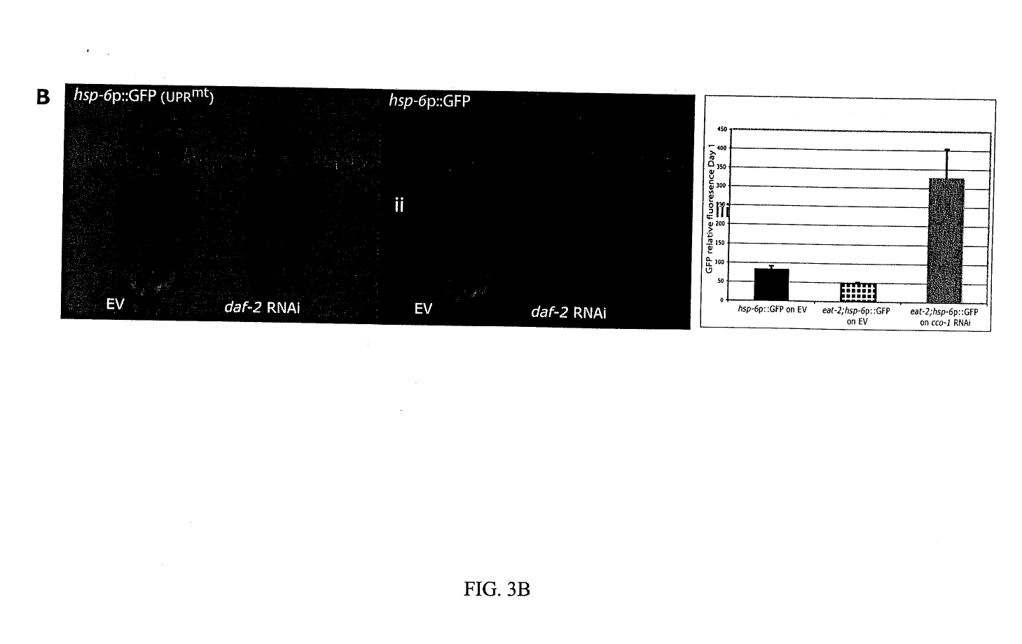

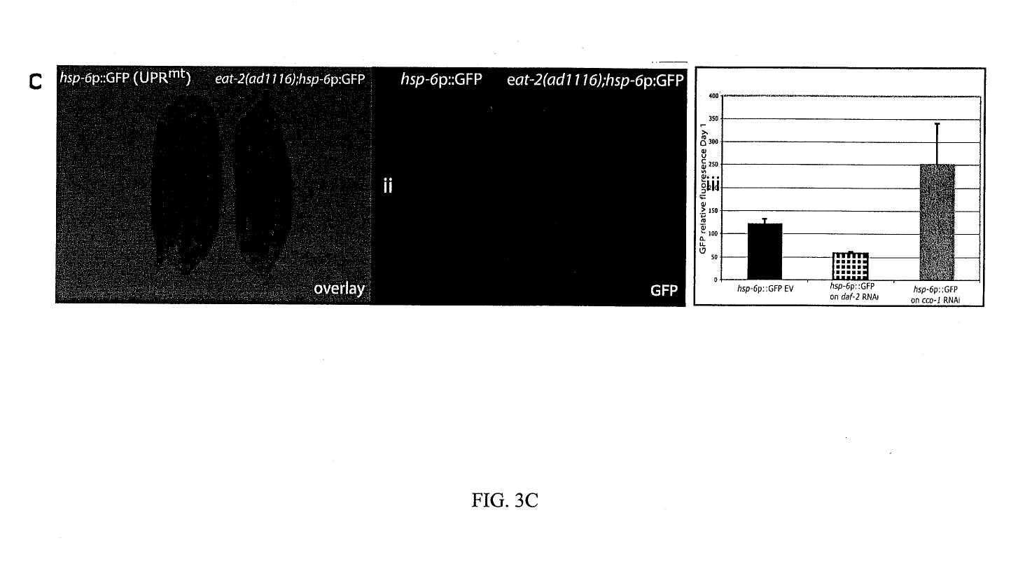

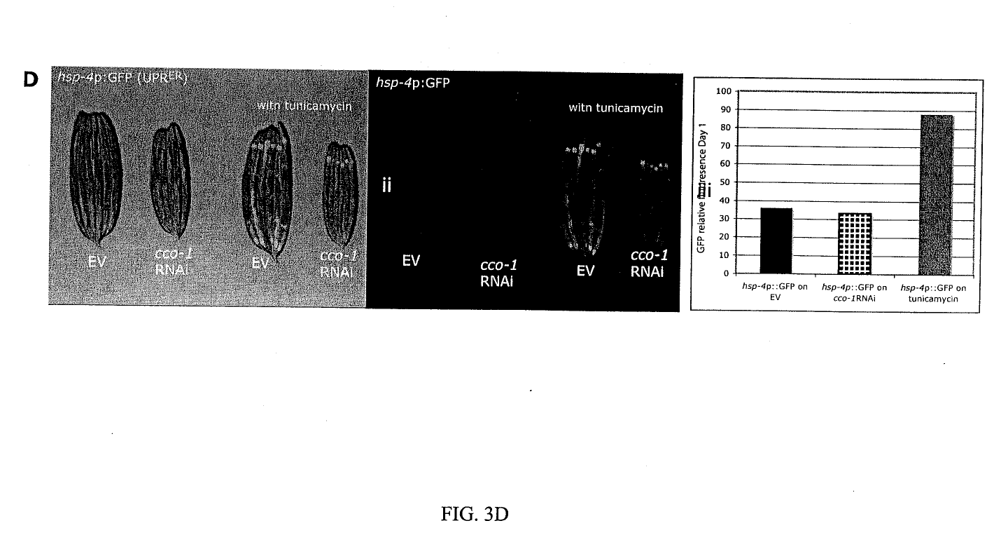

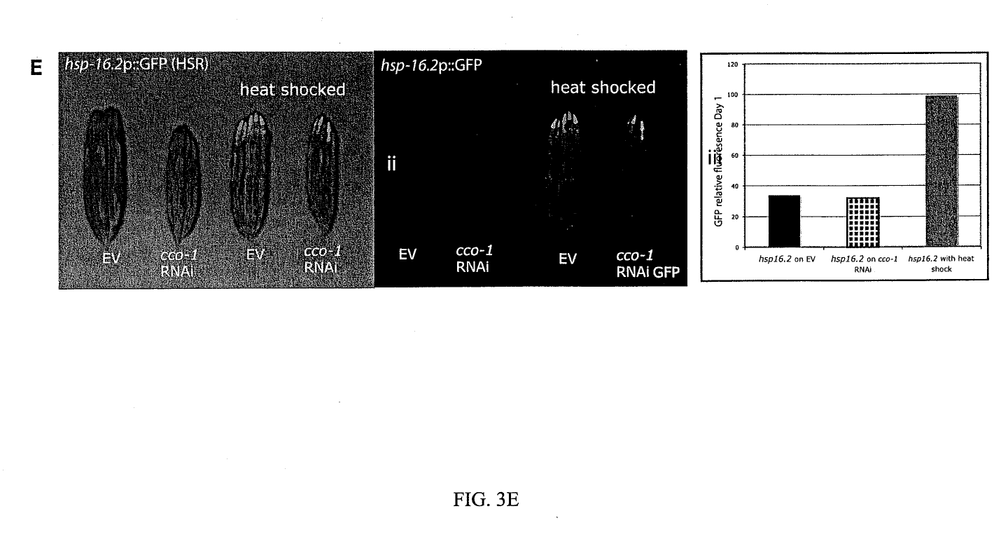

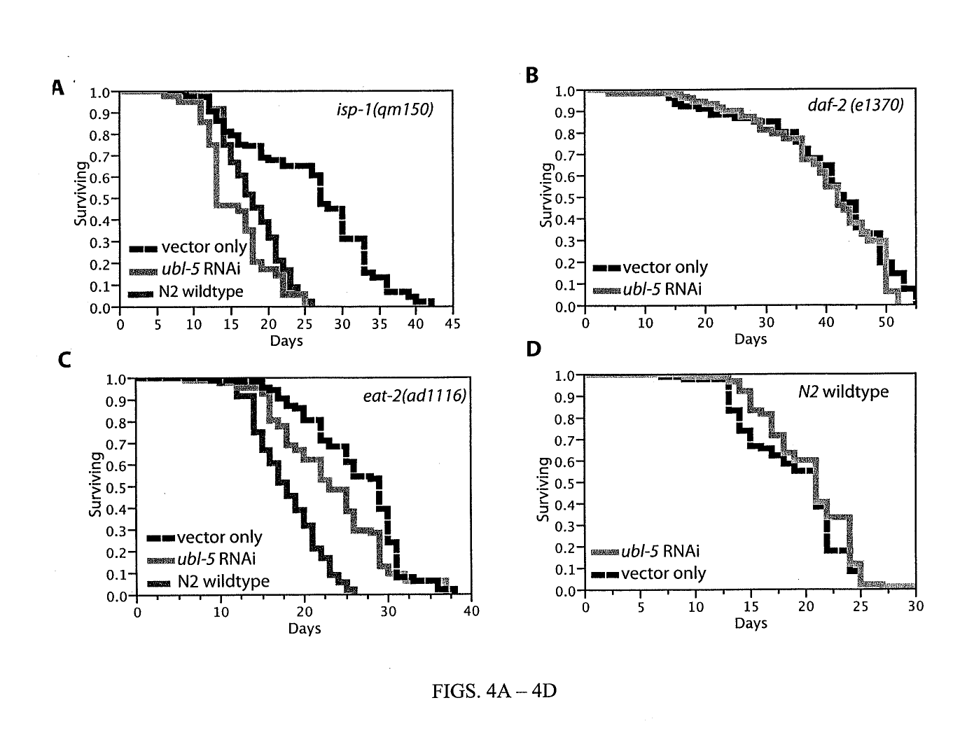

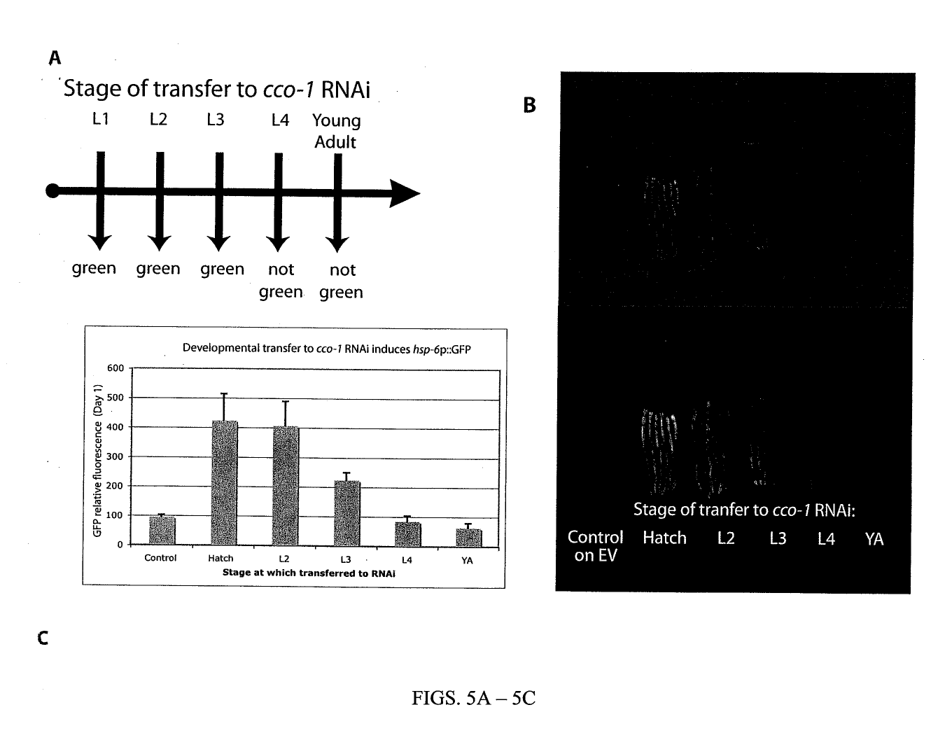

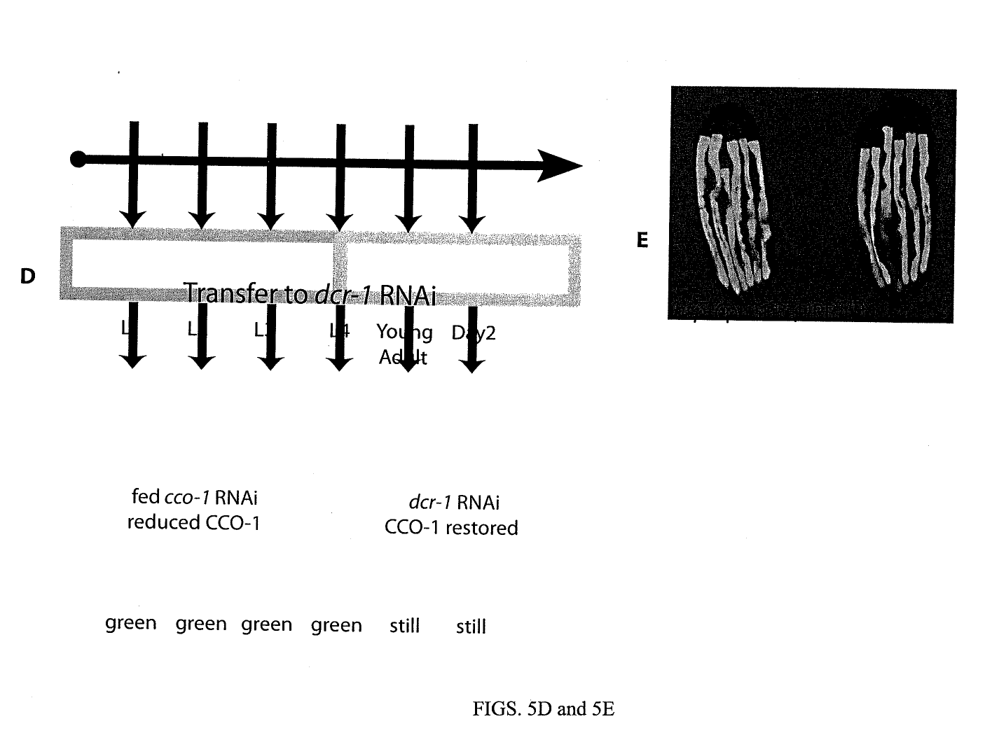

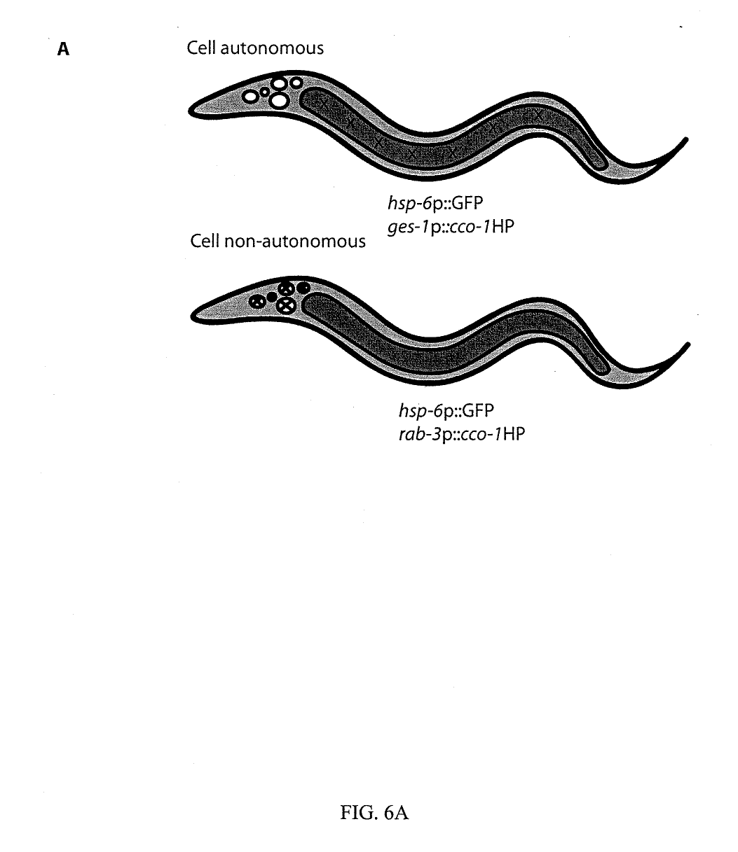

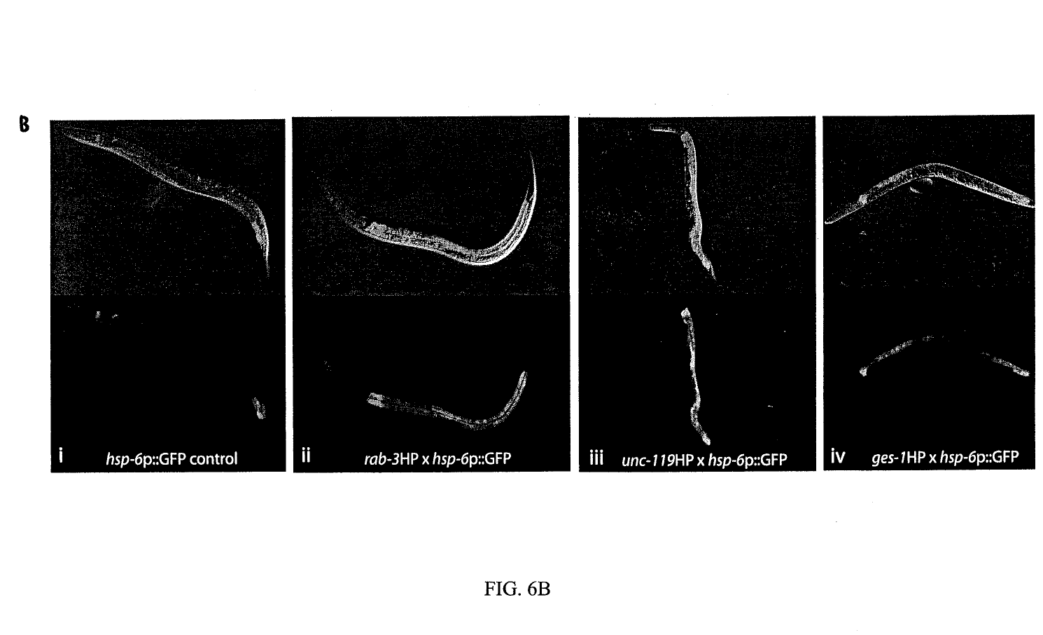

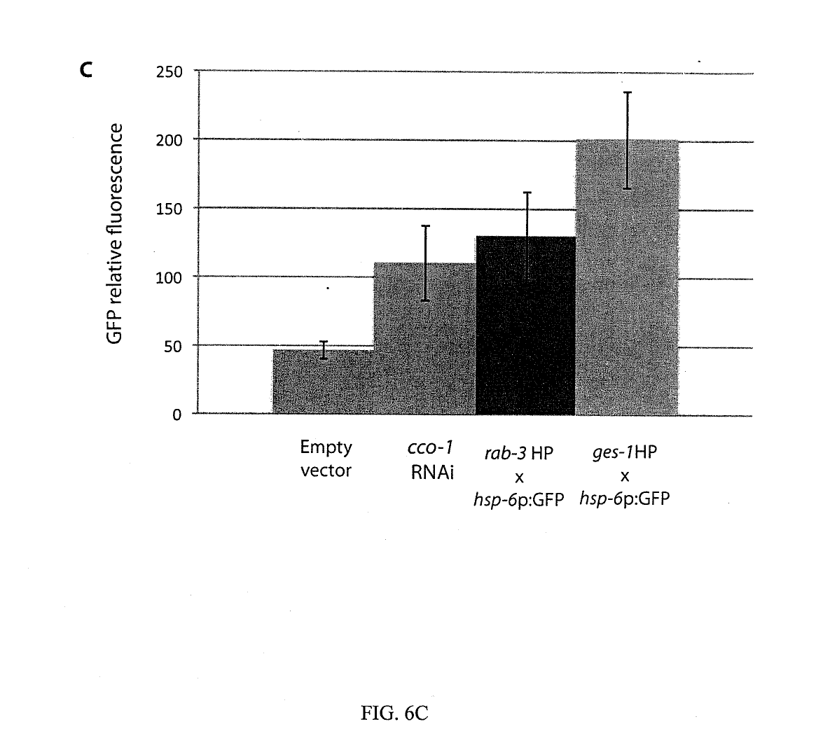

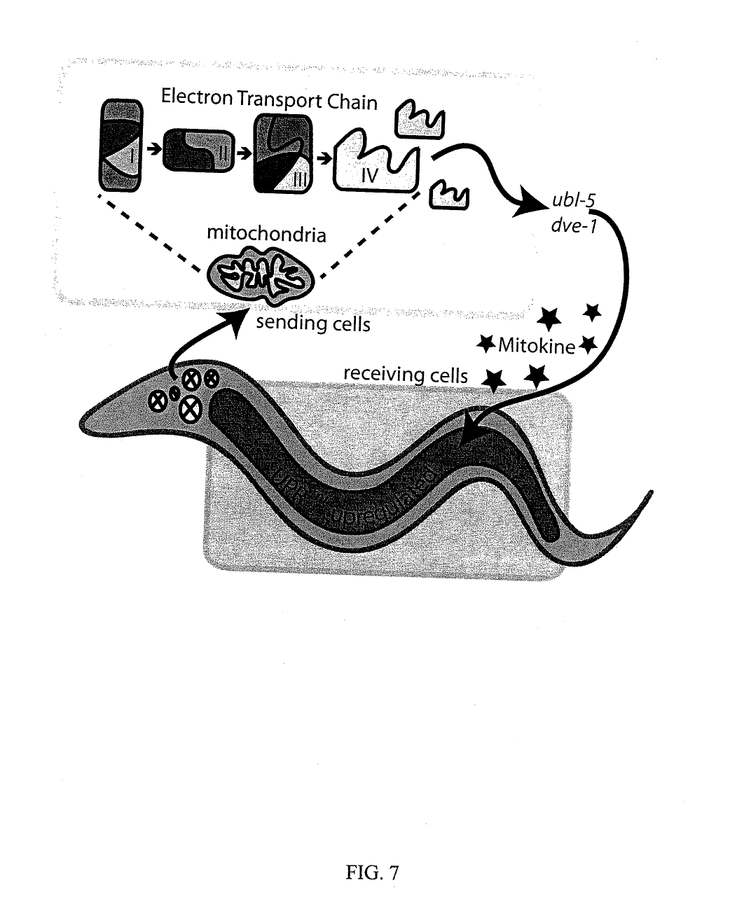

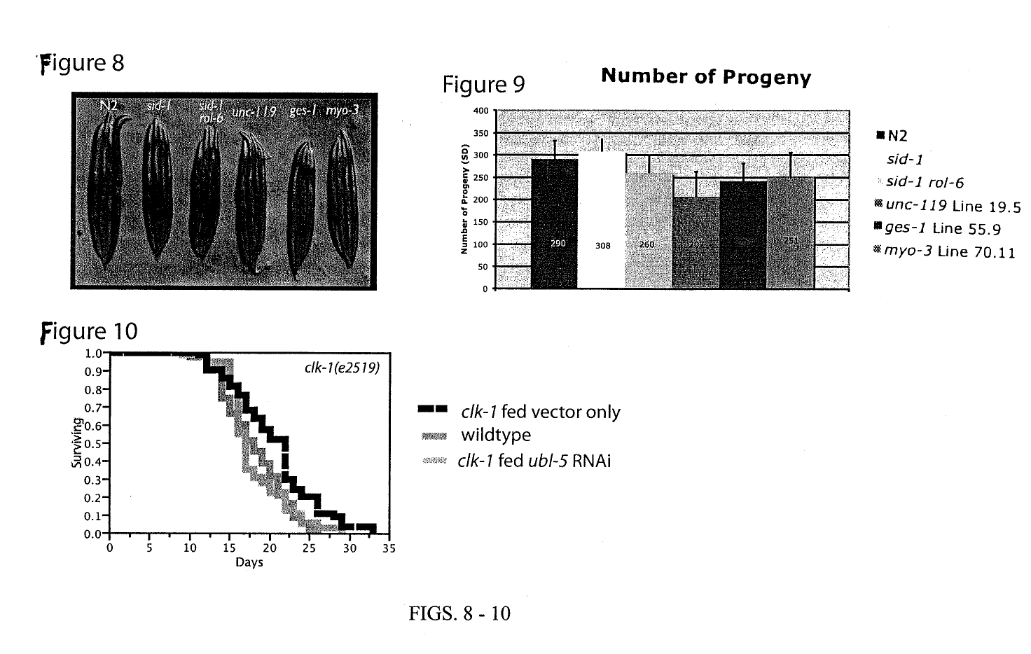

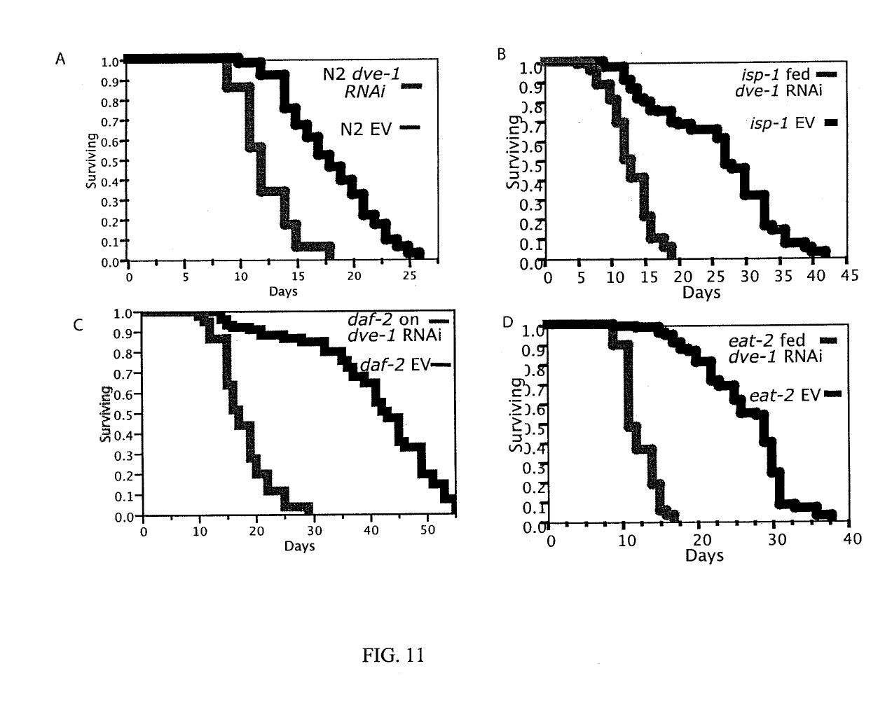

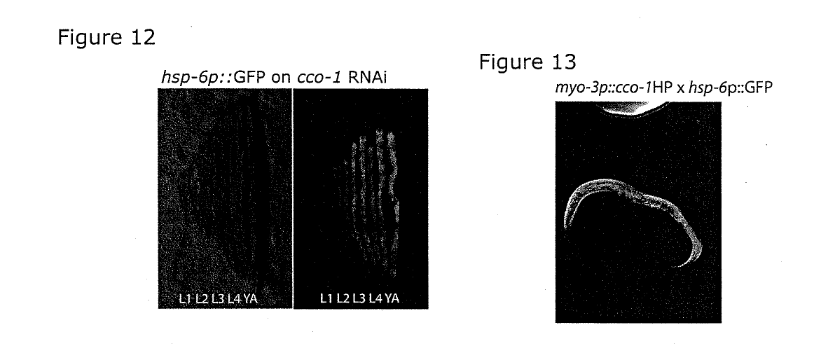

In worms and plants, RNAi can have a systemic effect because the dsRNA molecules are not maintained locally, but rather spread throughout the entire organism. For example, exposure of the intestine to bacterially expressed dsRNA results in the dsRNA entering through the intestinal lumen but eliciting knockdown in cells outside the intestine, such as the muscle and hypodermis (Jose et al., 2009). Therefore, it is probable that dsRNA expressed in one cell type could be exported and affects neighboring cells. To remove the systemic nature of RNAi from the experimental design, Applicants used systemic RNAi deficient sid-1(qt9) mutant worms (FIG. 1A). sid-1 encodes a transmembrane protein predicted to serve as a channel for dsRNA entry. While defective for systemic RNAi, the sid-1(qt9) mutants are fully functional for cell autonomous RNAi (Winston et al., 2002). Lines were generated in the sid-1(qt9) mutant background using an inverted repeat of the cco-1 cDNA under the control of well-characterized promoters expressed in neurons (unc-119 and rab-3) (Maduro and Pilgrim, 1995; Nonet et al., 1997), intestine (ges-1) (Aamodt et al., 1991), and body wall muscle cells (myo-3) (Miller et al., 1986; Okkema et al., 1993).

Utilizing this approach, knockdown of cco-1 in the intestine using the ges-1 intestinal-specific promoter driving a cco-1 hairpin construct significantly increased lifespan (FIG. 1B, representative line of 13, Table 1 and Table 5), whereas the myo-3 muscle-specific promoter driven cco-1 hairpin in the body wall muscle either had no effect or even decreased lifespan (FIG. 1C, representative line of 6, Table 1 and Table 5). The neuronal rab-3 promoter driven cco-1 hairpin also increased lifespan (FIG. 1D, representative line of 2, Table 1 and Table 5). Because the lifespan extension in the neuronal promoter line was not as great as that observed in intestinal hairpin lines, Applicants tested another neuronal promoter, the pan-neural unc-119 promoter. Consistent with the rab-3 promoter, Applicants observed a moderate increase in lifespan in multiple unc-119 transgenic lines (FIG. 1E, representative line of 8, Table 1 and Table 5). The results of these experiments suggest a primary requirement for ETC knockdown in intestinal and neuronal tissues for increased longevity, albeit the neuronal derived ETC knockdown was consistently less robust compared to the intestinal knockdown. Consistent with the hypothesis that the mitochondrial longevity pathway has a tissue-specific component, Applicants find that lifespan extension can be achieved through knockdown in one or more tissues, and knockdown of cco-1 in muscle cells reveals a potentially unknown lifespan shortening phenotype. Applicants observed robust results with the tissue-specific dsRNA hairpin approach, but sought to test an alternative method for tissue-specific RNAi. Tissue-specific RNAi can also be achieved by feeding dsRNA to rde-1 mutant animals in which the wildtype rde-1 gene has been rescued using tissue specific promoters (FIG. 2A) (Qadota et al., 2007). rde-1 encodes an essential component of the RNAi machinery encoding a member of the PIWI/STING/Argonaute family of proteins. Tissue specific gene knockdown using bacteria expressing dsRNA can be achieved by expressing a rde-1 rescue construct driven by a tissue-specific promoter (Qadota et al., 2007).

Lifespan analysis was performed with rde-1(ne219) mutant animals in which rde-1 was restored by tissue specific expression of wildtype rde-1 cDNA (Qadota et al., 2007). rde-1 was rescued in transgenic lines under the control of the lin-26 hypodermal promoter, the hlh-1 body wall muscle promoter, and the nhx-1 intestinal expressing promoter (Qadota et al., 2007). These lines were then tested for their effects on lifespan when animals were fed cco-1 dsRNA producing bacteria.

As expected, feeding rde-1(ne219) mutant animals cco-1 dsRNA producing bacteria did not extend lifespan, since these animals fail to perform RNAi due to the lack of rde-1 (FIG. 2B and Table 1). Consistent with the cco-1 hairpin approach, knockdown of cco-1 in the intestine, by the nhx-1p::rde-1 transgene, significantly increased lifespan when fed cco-1 dsRNA bacteria. In fact, intestinal cco-1 dsRNA was able to completely recapitulate the lifespan extension generated by feeding cco-1 dsRNA in wildtype animals (FIG. 2C and Table 1). Furthermore, cco-1 knockdown in the body wall muscle decreased lifespan (FIG. 2D and Table 1), similar to results obtained from the muscle specific cco-1 RNAi hairpin experiments. The hypodermal knockdown had no significant effect on lifespan (FIG. 2E and Table 1). Consistent with cco-1 feeding RNAi increasing longevity in an insulin/IGF-1 pathway independent manner, Applicants found the lifespan extension of intestinal cco-1 RNAi animals to be daf-16 independent (FIG. 2F and Table 1).

The results from the tissue specific ceo-1 hairpin experiments and the rde-1 tissue specific complementation experiments suggest that the knockdown of cco-1 in the intestine and nervous system is sufficient for the initiation of a tissue non-autonomous regulation of the aging process.

### Example 2

### Demonstrating that Tissue Specific ETC Knockdown Uncouples Multiple Correlates of Longevity

The aim of this example is to show tissue specific ETC knockdown uncouples multiple correlates of longevity.

Resistance to oxidative stress, UV damage and heat stress is associated with multiple forms of increased longevity. Applicants tested whether the increased longevity of tissue specific cco-1 RNAi animals was due to resistance to these stresses. Applicants found that tissue specific knockdown of cco-1 did not affect the response of animals to oxidative stress induced by paraquat in a manner correlated with their longevity phenotype (Table 2), consistent with recent results in worms and flies (Copeland et al., 2009; Doonan et al., 2008; Van Raamsdonk and Hekimi, 2009) (Lee et al., 2002). Furthermore, there is not a consistent correlation between long-lived flies with reduced ETC function and free-radical stress resistance (Copeland et al., 2009). Applicants next tested whether increased resistance to UV damage correlated with increased longevity. Again, Applicants found that none of the long-lived tissue specific hairpin lines were more resistant to UV damage than wild type animals (Table 3). Finally, Applicants tested whether the long-lived tissue specific cco-1 hairpin lines were more resistant to heat stress than control animals and found that they were not (Table 4). Collectively, the increased longevity of tissue specific cco-1 hairpin animals did not correlate with the known stress resistance phenotypes associated with other pathways that regulate the aging process (Arantes-Oliveira et al., 2002; Larsen et al., 1995; Lee et al., 1999; Lee et al., 2003; McElwee et al., 2004).

RNAi of cco-1 slows development, growth, movement and reduces fecundity (Dillin et al., 2002b). Through an RNAi dilution approach of nuclear encoded mitochondrial genes many of these side effects could be uncoupled from longevity and suggested a quantitative model for ETC function upon these life history traits (Rea et al., 2007). Applicants asked if there could also be a qualitative difference among the mitochondrial ETC from different tissues that could also explain the observed side effects of increased longevity by reduced ETC function. Applicants found that many of these traits could be uncoupled from increased longevity conferred by simply reducing mitochondrial function in a particular tissue. For example, long lived animals in which cco-1 was knocked down in the neuronal cells produced worms of nearly identical length to their control counterparts (FIG. 8), that reached adulthood at the similar rates (data not shown) and had similar number of progeny (FIG. 9). These results are consistent with ETC reduction in all tissues of Drosophila increased lifespan and decreased fertility, while knockdown in neurons increased longevity without affecting fertility (Copeland et al., 2009). Additionally, reduction of cco-1 in the intestine or nervous system did not result in slowed movement; however, reduction in the body wall muscles did. Therefore, in addition to the quantitative model proposed by Rea et al. to explain the developmental and behavioral deficits of ETC RNAi, contributions from specific tissues must also play an important role in these life history traits.

### Example 3

### The mitochondrial Unfolded Protein Response (UPRmt) and its Role in the ETC Longevity Pathway

The aim of this example is to show that UPRmt plays a central and specific role in the increased longevity generated by ETC RNAi.

In response to a mitochondrial perturbation there exists a stress response mechanism that is communicated to the nucleus to up regulate the expression of mitochondrial associated protein chaperones, such as HSP-6 and HSP-60, the mitochondrial-specific unfolded protein response (UPRmt) (Benedetti et al., 2006; Yoneda et al., 2004; Zhao et al., 2002). hsp-6 is the worm mitochondrial hsp10/60 chaperonin family member and hsp-60 is the worm mitochondrial hsp70 heat shock protein family member. This stress response pathway is activated upon sensing misfolding of mitochondrial specific proteins or stoichiometric abnormalities of large multimeric complexes, such as ETC complexes (Yoneda et al., 2004). Disrupting subunits of ETC complexes by either RNAi or mutation activates the mitochondrial stress response (Benedetti et al., 2006; Yoneda et al., 2004). Previously, cco-1 RNAi was found to be a potent inducer of hsp-6 and hsp-60 (Yoneda et al., 2004). Intrigued by this discovery, Applicants tested whether the UPRmt might play a central and specific role in the increased longevity generated by ETC RNAi.

Applicants tested whether other well known pathways that regulate the aging process resulted in the upregulation of the UPRmt. Unlike cco-1 RNAi treated animals (FIG. 3A), animals treated with RNAi towards daf-2, the IIS receptor or, eat-2 mutant animals, a genetic surrogate for diet restriction induced longevity, did not induce the UPRmt marker hsp-6p::GFP (FIGS. 3B and C, respectively) even though each of these interventions increase longevity. Therefore, induction of the UPRmt appears specific to the ETC longevity pathway and not other longevity pathways.

In addition to the UPRmt, the canonical unfolded protein response in the endoplasmic reticulum (UPRER) is also induced under conditions of protein misfolding, although confined to the ER (Ron and Walter, 2007). Applicants tested whether mitochondrial reduction resulted in a general up regulation of all protein misfolding pathways by treating hsp-4-p-4::GFP reporter worm strains with cco-1 RNAi. HSP-4 is the worm orthologue of the ER chaperone, BiP, which is transcriptionally induced by the UPRER. Unlike the UPRmt, cco-1 RNAi did not induce expression of the UPRER reporter (FIG. 3D), although ER stress induced by tunicamycin did. Furthermore, cco-1 RNAi did not inhibit the ability of cells to induce the UPRER by treatment with tunicamycin. Applicants also tested if cco-1 RNAi induced a marker of cytosolic protein misfolding by treating animals containing the hsp-16.2p::GFP reporter strain with cco-1 RNAi. HSP-16.2 is a worm small heat shock protein of the hsp20/alpha-B crystallin family and is under transcriptional control of the Heat Shock Response (HSR) predominantly regulated by HSF-1. Much like the UPRER, cco-1 RNAi was unable to induce this reporter associated with cytosolic misfolding (FIG. 3E). As positive controls, heat shock could induce the HSR reporter and cco-1 RNAi did not block this response. Thus, it appears that knockdown of cco-1 specifically induces the UPRmt, and not other protein misfolding pathways.

### Example 4

### The UPRmt is a Potent Transducer of the ETC Longevity Pathway

The aim of this example is to show that UPRmt is a potent transducer of the ETC longevity pathway.

Applicants tested whether the UPRmt is a key component of the ETC longevity pathway since there appeared to be a positive and specific correlation of induction of the UPRmt and ETC mediated longevity. If the UPRmt is indeed a regulator of the ETC longevity pathway, the loss of the UPRmt function would be expected to specifically suppress the extended longevity of animals caused by ETC RNAi and not other longevity pathways.

The UPRmt consists of a signaling cascade that results in upregulation of nuclear encoded genes to alleviate the stress sensed in the mitochondria. Perception of misfolding in the mitochondria requires the nuclear localized ubiquitin-like protein UBL-5, which acts as an important and specific coactivator of the homeodomain transcription factor, DVE-1. Together, UBL-5 and DVE-1 respond to mitochondrial perturbation to increase expression of mitochondrial chaperones, including hsp-6 and hsp-60 (Benedetti et al., 2006). ClpP is the homolog of the E. coli ClpP protease located in the mitochondria that plays a role in generating the mitochondrial derived signal to up regulate DVE-1/UBL-5 stress responsive genes (Haynes et al., 2007). The targets and mode of ClpP activation are unknown.

Applicants treated long-lived ETC mutant animals with RNAi directed towards the known pathway components of the UPRmt and tested the resulting lifespan. Applicants found that RNAi of ubl-5, the dve-1 transcriptional co-regulator, specifically blocked the extended lifespan of the mitochondrial mutants, isp-1 (qm150) and clk-1(e2519) (FIGS. 4A and 10, respectively, Table 1) compared to the lifespan of wildtype animals. RNAi of ubl-5 did not suppress the extended lifespan of long-lived daf-2 or eat-2 mutant animals (FIGS. 4B and C, respectively, Table 1). Furthermore, ubl-5 RNAi did not shorten the lifespan of wild type animals (FIG. 4D and Table 1). Taken together, ubl-5 appears essential and specific for the extended longevity of mitochondrial mutants.

RNAi of dve-1 suppressed the lifespan of all long-lived animals and shortened the lifespan of wild type animals (FIG. 11A-D). This result is not surprising given the roles of dve-1 in growth and development and the embryonic lethality observed for homozygous dve-1 mutant animals (Burglin and Cassata, 2002; Haynes et al., 2007). Furthermore, RNAi of hsp-6, hsp-60 or clpp-1 suppressed longevity in the same manner as dve-1, suggesting that these RNAi treatments were pleiotropic and simply made the animals sick. Thus, results indicate that ubl-5 is specific for the longevity response, possibly by specifying the transcriptional activity of DVE-1, to mitochondrial ETC mediated longevity and dye-1, hsp-6, hsp-60 and clpp-1 have more broad roles in development and survival that make their specific roles in mitochondrial ETC mediated longevity difficult to discern at this time.

### Example 5

### The Temporal Requirements of the UPRmt and ETC Mediated Longevity Overlap

The aim of this example is to show the temporal requirements of the UPRmt and ETC mediated longevity overlap

The lifespan extension by ETC RNAi has a distinct temporal requirement during the L3/L4 stages of larval development (Dillin et al., 2002b; Rea et al., 2007). Furthermore, markers of the UPRmt, namely hsp-6p::GFP have their greatest activation late in larval development at the L4 stage when challenged with mitochondrial stress (Yoneda et al., 2004). Applicants verified these findings by following the activation of the UPRmt of animals treated with cco-1 RNAi (FIG. 12) and asked whether the timing requirement of cco-1 mediated longevity could be uncoupled from the induction of the UPRmt. Worms carrying the hsp-6p::GFP UPRmt reporter were transferred onto bacteria expressing cco-1 dsRNA at every developmental stage from embryo to day 2 of adulthood (FIGS. 5A and B). Applicants found that worms could induce the UPRmt, as observed by increased GFP fluorescence, if transferred to the cco-1 RNAi treatment before the L4 larval stage (FIGS. 5B and C). After the LA larval stage, worms transferred to bacteria expressing cco-1 dsRNA were unable to induce the hsp-6p::GFP marker (FIGS. 5B and C) and were not long lived (Dillin et al., 2002b; Rea et al., 2007). Thus, inactivation of cco-1 must be instituted before the L3/L4 larval stage to initiate induction of the UPRmt. Inactivation in adulthood does not induce the UPRmt and does not result in increased longevity (FIG. 5A-C). Taken together, the induction of increased longevity and the UPRmt must occur prior to the L3/L4 larval stages and this induction cannot be temporally uncoupled from each other. Inactivation of ETC components during larval development is sufficient to confer increased longevity on adult animals even though the knocked-down ETC component can be restored in adulthood, suggesting that a longevity signal is initiated during development and maintained well into adulthood (Dillin et al., 2002b; Rea et al., 2007). Applicants tested whether developmental inactivation of cco-1 could not only induce, but whether it could also maintain activation of the UPRmt during adulthood, even though adult inactivation of cco-1 was unable to induce the UPRmt (FIG. 5A-C). Worms treated with cco-1 RNAi during larval development and then moved to dicer (dcr-1) RNAi (a key component of the RNAi machinery) to block further RNAi activity on day 1 of adulthood have an extended lifespan (Dillin et al., 2002b; Rea et al., 2007). Similarly, hsp-6p::GFP worms treated with cco-1 RNAi during larval development and moved onto dcr-1 RNAi maintained the induced response of the UPRmt (FIG. 5E). Therefore, inactivation during larval development of cco-1 is sufficient to initiate and maintain a signal to increase longevity and induce the UPRmt in adult animals. The results of these experiments match the timing requirements of the lifespan extension for ETC RNAi treated worms and support the idea that the signals for increased longevity and induction/maintenance of the UPRmt are not separable, suggesting that the UPRmt could be responsible, at least in part, for the signaling event that sets and maintains the rate of aging for the adult animal.

### Example 6

### The UPRmt Responds to Cell Non-Autonomous Cues from ETC Knockdown

The aim of this example is to show that UPRmt responds to cell non-autonomous cues from ETC knockdown.

Intrigued by the tissue specific nature by which cco-1 depletion can control the aging process of the entire animal, the specific role of the UPRmt in the longevity response in ETC mutant animals and the overlapping timing requirements for both ETC RNAi and induction of the UPRmt, Applicants hypothesized that the induction of the UPRmt may be able to act cell non-autonomously in a multicellular organism. If so, Applicants reasoned that induction of the UPRmt in one tissue by cco-1 reduction might lead to the UPRmt being upregulated in a distal tissue that has not experienced cco-1 reduction (FIG. 6A). Consistent with this hypothesis, transgenic worms with the cco-1 hairpin expressed in all neurons (either the rab-3 or the unc-119) promoter driven cco-1 hairpin) were able to induce hsp-6p:GFP expression in the intestine (FIGS. 6Bii and iii). In fact, neuronal RNAi of cco-1 induced the UPRmt reporter to the same extent as animals with intestinal cco-1 RNAi (FIGS. 6B ii-iv and C). Applicants were unable to ascertain whether mitochondrial ETC knockdown in the intestine could signal to the nervous system to induce the UPRmt due to the low expression of the hsp-6p::GFP reporter in neuronal cells. It is unclear if hsp-6 is not expressed in neurons or at a level thus far undetected by the reporter construct. However, it is clear that mitochondrial disruption in one tissue can be sensed and a stress response signal can be generated to communicate to other cells and tissues to coordinate an organism wide response to mitochondrial challenges.

### Example 7

### Methods and Materials

Strains: HC114 (sid-1(qt9)), MQ887 (isp-1(qm150)), CB4876 (clk-1(e2519)), CF1041 (daf-2(e1370)), TK22 (mev-1(kn1)III), WB27 (rde-1(ne219)), NR222 (rde-1(ne219)V; kzIs9, NR350 (rde-1(ne219) V; kzIs20), SJ4100 (zcIs13[hsp-6p::GFP]), SJ4058 (zcIs9[hsp-60p::GFP]), CL2070 (dvIs[hsp-16.2::GFP]) and N2 wildtype were obtained from the Caenorhabditis Genetics Center. VP303 was a generous gift from the Strange lab.

The myo-3 promoter hairpin RNAi transgene was created by inserting PCR amplified cco-1 cDNA with no stop codon into pPD97.86 (Addgene). The reverse complement cco-1 cDNA was inserted into pGEX2T after the GST linker to be used as the hairpin loop as described (Tavernakis 2001). PCR amplifications were used to add an AgeI site to the 3′ end of the cco-1 cDNA and NgoMIV (compatible and non-receivable with AgeI) to the 5′ end of the GST linker. Ligation of the PCR products in the presence of AgeI enzyme and NgoMIV were followed by gel extraction of the promoter hairpin fragment as described (Hobert 2002). The ges-1 and unc-119 promoters were PCR amplified from genomic DNA and cloned in place of the myo-3 promoter driving cco-1. The rab-3 promoter was a gift from Kang Shen, Stanford University, and sequence verified.

Transgenic tissue-specific RNAi hairpin expressing strains were generated by microinjecting gel extracted hairpin RNAi constructs (40-60 ng/μl) mixed with an equal concentration of pRF4(rol-6) co-injection marker or myo-2::GFP into sid-1(qt9) worms. Control lines were generated by injecting sid-1(qt9) with 50 ng/μl pRF4(rol-6). Extrachromosomal arrays were integrated and backcrossed 5 times as described (Hope 1999).

Lifespan Analyses: Lifespan analyses were performed as described previously (Dillin 2002). 80-100 animals were used per condition and scored every day or every other day. All lifespan analyses were conducted at 20° C. JMP IN 8 software was used for statistical analysis. In all cases, P-values were calculated using the log-rank (Mantel-Cox) method.
GFP Expression and Quantification: SJ4100 hsp-6::GFP were bleached to collect synchronous eggs and grown on cco-1 RNAi. At each stage from larval stage 1 to Day 1 of adulthood, worms were assayed for GFP expression. Alternatively, SJ4100 worms were grown on empty vector and transferred to cco-1 RNAi at each developmental stage at which time GFP was assayed at Day 1 or 2 of adulthood.
Integrated hairpin RNAi worm lines were crossed to SJ4100 hsp-6p::GFP reporter lines. GFP was monitored in Day 1 adults. Fluorimetry assays were performed using a Tecan fluorescence plate reader. 100 roller worms were picked at random (25 into 4 wells of a black walled 96-well plate) and each well was read three times and averaged. Each experiment was repeated three times.
Stress Assays: Paraquat assays were performed as described (Dillin 2002). For UV irradiation assays, worms were grown to day 5 of adulthood. Worms were then transferred to plates without food and exposed to 1200 J/m2 of UV using an UV Stratalinker. Worms were transferred back to seeded plates and scored daily for viability. For heat-shock assays, worms were grown to day 1 of adulthood. Worms were then transferred to plates without food and placed at 33° C. Worms were checked every 2 hr for viability.
Reproductive assays: Animals were synchronized. Gravid adults were allowed to lay eggs on a seeded plate. ˜8-10 hours later larvae were picked to new individual plates as they hatched within 10 minute period. The fecundity of 30 animals/genotype were monitored by placing 1 animal on a plate and transferring every 12 hours to new plate. The resulting progeny were allowed to grow to adulthood and were counted.

### REFERENCES

Aamodt, E. J., Chung, M. A., and McGhee, J. D. (1991). Spatial control of gut-specific gene expression during Caenorhabditis elegans development. Science 252, 579-582.
Apfeld, J., and Kenyon, C. (1998). Cell nonautonomy of C. elegans daf-2 function in the regulation of diapause and life span. Cell 95, 199-210.
Arantes-Oliveira, N., Apfeld, J., Dillin, A., and Kenyon, C. (2002). Regulation of Life-Span by Germ-Line Stem Cells in Caenorhabditis elegans. Science 295, 502-505.
Benedetti, C., Haynes, C. M., Yang, Y., Harding, H. P., and Ron, D. (2006). Ubiquitin-like protein 5 positively regulates chaperone gene expression in the mitochondrial unfolded protein response. Genetics 174, 229.
Bishop, N. A., and Guarente, L. (2007). Two neurons mediate diet-restriction-induced longevity in C. elegans. Nature 447, 545-549.
Bluher, M., Kahn, B. B., and Kahn, C. R. (2003). Extended Longevity in Mice Lacking the Insulin Receptor in Adipose Tissue. Science 299, 572-574.
Broughton, S. J., Piper, M. D. W., Ikeya, T., Bass, T. M., Jacobson, J., Driege, Y., Martinez, P., Hafen, E., Withers, D. J., and Leevers, S. J. (2005). Longer lifespan, altered metabolism, and stress resistance in Drosophila from ablation of cells making insulin-like ligands. Proceedings of the National Academy of Sciences 102, 3105.
Burglin, T. R., and Cassata, G. (2002). Loss and gain of domains during evolution of cut superclass homeobox genes. International Journal of Developmental Biology 46, 115-124.
Conboy, I. M., Conboy, M. J., Wagers, A. J., Girma, E. R., Weissman, I. L., and Rando, T. A. (2005). Rejuvenation of aged progenitor cells by exposure to a young systemic environment. Nature 433, 760-764.
Copeland, J. M., Cho, J., Lo, T., Hur, J. H., Bahadorani, S., Arabyan, T., Rabie, J., Soh, J., and Walker, D. W. (2009). Extension of Drosophila Life Span by RNAi of the Mitochondrial Respiratory Chain. Current Biology.
Dell'Agnello, C., Leo, S., Agostino, A., Szabadkai, G., Tiveron, C., Zulian, A., Prelle, A., Roubertoux, P., Rizzuto, R., and Zeviani, M. (2007). Increased longevity and refractoriness to Ca2+-dependent neurodegeneration in Surf1 knockout mice. Human Molecular Genetics 16, 431.
Dillin, A., Crawford, D. K., and Kenyon, C. (2002a). Timing Requirements for Insulin/IGF-1 Signaling in C. elegans. Science 298, 830-834.
Dillin, A., Hsu, A.-L., Arantes-Oliveira, N., Lehrer-Graiwer, J., Hsin, H., Fraser, A. G., Kamath, R. S., Ahringer, J., and Kenyon, C. (2002b). Rates of Behavior and Aging Specified by Mitochondrial Function During Development. Science 298, 2398-2401.
Doonan, R., McElwee, J. J., Matthijssens, F., Walker, G. A., Houthoofd, K., Back, P., Matscheski, A., Vanfleteren, J. R., and Gems, D. (2008). Against the oxidative damage theory of aging: superoxide dismutases protect against oxidative stress but have little or no effect on life span in Caenorhabditis elegans. Genes & Development 22, 3236.
Feng, J., BussiËre, F., and Hekimi, S. (2001). Mitochondrial electron transport is a key determinant of life span in Caenorhabditis elegans. Developmental Cell 1, 633-644.
Giannakou, M., E., Goss, M., and Partridge, L. (2008). Role of dFOXO in lifespan extension by dietary restriction in <i>Drosophila melanogaster</i>: not required, but its activity modulates the response. Aging Cell 7, 187-198.
Golden, T. R., Beckman, K. B., Lee, A. H. J., Dudek, N., Hubbard, A., Samper, E., and Melov, S. (2007). Dramatic age-related changes in nuclear and genome copy number in the nematode Caenorhabditis elegans. Aging Cell 6, 179.
Hansen, M., Chandra, A., Mitic, L. L., Onken, B., Driscoll, M., and Kenyon, C. (2008). A role for autophagy in the extension of lifespan by dietary restriction in C. elegans. PLoS Genet 4, e24.
Harman, D. (1956). Aging: A Theory Based on Free Radical and Radiation Chemistry. J Gerontol
   11, 298-300.
Haynes, C. M., Petrova, K., Benedetti, C., Yang, Y., and Ron, D. (2007). ClpP mediates activation of a mitochondrial unfolded protein response in C. elegans. Developmental Cell 13, 467-480.
Herndon, L. A., Schmeissner, P. J., Dudaronek, J. M., Brown, P. A., Listner, K. M., Sakano, Y., Paupard, M. C., Hall, D. H., and Driscoll, M. (2002). Stochastic and genetic factors influence tissue-specific decline in ageing C. elegans. Nature 419, 808-814.
Hwangbo, D. S., Gersham, B., Tu, M.-P., Palmer, M., and Tatar, M. (2004). Drosophila dFOXO controls lifespan and regulates insulin signalling in brain and fat body. Nature 429, 562-566.
Iser, W. B., Gami, M. S., and Wolkow, C. A. (2007). Insulin signaling in Caenorhabditis elegans regulates both endocrine-like and cell-autonomous outputs. Developmental biology 303, 434-447.
Jose, A. M., Smith, J. J., and Hunter, C. P. (2009). Export of RNA silencing from C. elegans tissues does not require the RNA channel SID-1. Proceedings of the National Academy of Sciences 106, 2283.
Kapahi, P., Zid, B. M., Harper, T., Koslover, D., Sapin, V., and Benzer, S. (2004). Regulation of lifespan in Drosophila by modulation of genes in the TOR signaling pathway. Current Biology 14, 885-890.
Kenyon, C., Chang, J., Gensch, E., Rudner, A., and Tabtiang, R. (1993). A C. elegans mutant that lives twice as long as wild type. Nature 366, 461-464.
Kirchman, P. A., Kim, S., Lai, C. Y., and Jazwinski, S. M. (1999). Interorganelle signaling is a determinant of longevity in Saccharomyces cerevisiae. Genetics 152, 179-190.
Kuznetsov, A. V., Hermann, M., Saks, V., Hengster, P., and Margreiter, R. (2009). The cell-type specificity of mitochondrial dynamics. The International Journal of Biochemistry & Cell Biology 41, 1928-1939.
Kwong, L. K., and Sohal, R. S. (2000). Age-related changes in activities of mitochondrial electron transport complexes in various tissues of the mouse. Archives of Biochemistry and Biophysics 373, 16-22.
Lapointe, J., Stepanyan, Z., Bigras, E., and Hekimi, S. (2009). Reversal of the mitochondrial phenotype and slow development of oxidative biomarkers of aging in long-lived Mclk1+/−mice. Journal of Biological Chemistry, M109.
Larsen, P. L., Albert, P. S., and Riddle, D. L. (1995). Genes that regulate both development and longevity in Caenorhabditis elegans. Genetics 139, 1567-1583.
Lee, C. K., Klopp, R. G., Weindruch, R., and Prolla, T. A. (1999). Gene expression profile of aging and its retardation by caloric restriction. Science 285, 1390.
Lee, S. S., Kennedy, S., Tolonen, A. C., and Ruvkun, G. (2003). DAF-16 Target Genes That Control C. elegans Life-Span and Metabolism. Science 300, 644-647.
Lee, S. S., Lee, R. Y. N., Fraser, A. G., Kamath, R. S., Ahringer, J., and Ruvkun, G. (2002). A systematic RNAi screen identifies a critical role for mitochondria in C. elegans longevity, nature genetics 33, 40-48.
Li, J., Ebata, A., Dong, Y., Rizki, G., Iwata, T., and Lee, S. S. (2008). Caenorhabditis elegans HCF-1 functions in longevity maintenance as a DAF-16 regulator. PLoS Biol 6, e233.
Libina, N., Berman, J. R., and Kenyon, C. (2003). Tissue-specific activities of C. elegans DAF-16 in the regulation of lifespan. Cell 115, 489-502.
Liu, X., Jiang, N., Hughes, B., Bigras, E., Shoubridge, E., and Hekimi, S. (2005). Evolutionary conservation of the clk-1-dependent mechanism of longevity: loss of mclk1 increases cellular fitness and lifespan in mice. Genes & development 19, 2424-2434.
Maduro, M., and Pilgrim, D. (1995). Identification and Cloning of unc-119, a Gene Expressed in the Caenorhabditis elegans Nervous System. Genetics 141, 977-988.
McElwee, J. J., Schuster, E., Blanc, E., Thomas, J. H., and Gems, D. (2004). Shared transcriptional signature in Caenorhabditis elegans Dauer larvae and long-lived daf-2 mutants implicates detoxification system in longevity assurance. Journal of Biological Chemistry 279, 44533.
Miller, D. M., Stockdale, F. E., and Karn, J. (1986). Immunological identification of the genes encoding the four myosin heavy chain isoforms of Caenorhabditis elegans. Proceedings of the National Academy of Sciences 83, 2305-2309.
Nonet, M. L., Staunton, J. E., Kilgard, M. P., Fergestad, T., Hartwieg, E., Horvitz, H. R., Jorgensen, E. M., and Meyer, B. J. (1997). Caenorhabditis elegans rab-3 Mutant Synapses Exhibit Impaired Function and Are Partially Depleted of Vesicles. J Neurosci 17, 8061-8073.
Ogg, S., Paradis, S., Gottlieb, S., Patterson, G. I., Lee, L., Tissenbaum, H. A., and Ruvkun, G. (1997). The Forkheadtranscription factor DAF-16 transduces insulin-like metabolic and longevitysignalsin C. elegans. Nature 389, 995.
Okkema, P. G., Harrison, S. W., Plunger, V., Aryana, A., and Fire, A. (1993). Sequence Requirements for Myosin Gene Expression and Regulation in Caenorhabditis elegans. Genetics 135, 385-404.
Panowski, S. H., Wolff, S., Aguilaniu, H., Durieux, J., and Dillin, A. (2007). PHA-4/Foxa mediates diet-restriction-induced longevity of C. elegans. Nature 447, 550-555.
Pearl, R. (1928). The Rate of Living (London, UK, University of London Press).
Qadota, H., Inoue, M., Hikita, T., K̂ppen, M., Hardin, J. D., Amano, M., Moerman, D. G., and Kaibuchi, K. (2007). Establishment of a tissue-specific RNAi system in C. elegans. Gene 400, 166-173.
Rea, S., and Johnson, T. E. (2003). A metabolic model for life span determination in Caenorhabditis elegans. Developmental cell 5, 197-203.
Rea, S. L., Ventura, N., and Johnson, T. E. (2007). Relationship between mitochondrial electron transport chain dysfunction, development, and life extension in Caenorhabditis elegans. PLoS Biol 5, e259.
Ron, D., and Walter, P. (2007). Signal integration in the endoplasmic reticulum unfolded protein response. Nature Reviews Molecular Cell Biology 8, 519-529.
Rossignol, R., Letellier, T., Malgat, M., Rocher, C., and Mazat, J. P. (2000). Tissue variation in the control of oxidative phosphorylation: implication for mitochondrial diseases. BIOCHEMICAL JOURNAL-LONDON-347, 45-53.
Rubner, M. (1908). Das Problem der Lebensdauer und seine Be-ziehungen zu Wachstum und ErnaË hrung (Muenchen, Germany, R. Oldenburg).
Russell, S. J., and Kahn, C. R. (2007). Endocrine regulation of ageing. Nature Reviews Molecular Cell Biology 8, 681-691.
Taguchi, A., Wartschow, L. M., and White, M. F. (2007). Brain IRS2 signaling coordinates life span and nutrient homeostasis. Science 317, 369.
Tatar, M., Bartke, A., and Antebi, A. (2003). The Endocrine Regulation of Aging by Insulin-like Signals. Science 299, 1346-1351.
Tsang, W. Y., Sayles, L. C., Grad, L. I., Pilgrim, D. B., and Lemire, B. D. (2001). Mitochondrial Respiratory Chain Deficiency in Caenorhabditis elegans Results in Developmental Arrest and Increased Life Span. J Biol Chem 276, 32240-32246.
Tullet, J. M. A., Hertweck, M., An, J. H., Baker, J., Hwang, J. Y., Liu, S., Oliveira, R. P., Baumeister, R., and Blackwell, T. K. (2008). Direct inhibition of the longevity-promoting factor SKN-1 by insulin-like signaling in C. elegans. Cell 132, 1025-1038.
Van Raamsdonk, J. M., and Hekimi, S. (2009). Deletion of the Mitochondrial Superoxide Dismutase sod-2 Extends Lifespan in Caenorhabditis elegans. PLoS Genetics 5.
Wessells, R. J., Fitzgerald, E., Cypser, J. R., Tatar, M., and Bodmer, R. (2004a). Insulin regulation of heart function in aging fruit flies. Nat Genet. 36, 1275-1281.
Wessells, R. J., Fitzgerald, E., Cypser, J. R., Tatar, M., and Bodmer, R. (2004b). Insulin regulation of heart function in aging fruit flies. Nature genetics 36, 1275-1281.
Winston, W. M., Molodowitch, C., and Hunter, C. P. (2002). Systemic RNAi in C. elegans Requires the Putative Transmembrane Protein SID-1. Science 295, 2456-2459.
Wolff, S., Ma, H., Burch, D., Maciel, G. A., Hunter, T., and Dillin, A. (2006). SMK-1, an Essential Regulator of DAF-16-Mediated Longevity. Cell 124, 1039-1053.
Wolkow, C. A., Kimura, K. D., Lee, M.-S., and Ruvkun, G. (2000). Regulation of C. elegans Life-Span by Insulinlike Signaling in the Nervous System. Science 290, 147-150.
Wong, A., Boutis, P., and Hekimi, S. (1995). Mutations in the clk-1 gene of Caenorhabditis elegans affect developmental and behavioral timing. Genetics 139, 1247-1259.
Yoneda, T., Benedetti, C., Urano, F., Clark, S. G., Harding, H. P., and Ron, D. (2004). Compartment-specific perturbation of protein handling activates genes encoding mitochondrial chaperones. J Cell Sci 117, 4055-4066.
Zhao, Q., Wang, J., Levichkin, I. V., Stasinopoulos, S., Ryan, M. T., and Hoogenraad, N.J. (2002). A mitochondrial specific stress response in mammalian cells. The EMBO Journal 21, 4411-4419.

Having thus described in detail embodiments of the present invention, it is to be understood that the invention defined by the above paragraphs is not to be limited to particular details set forth in the above description as many apparent variations thereof are possible without departing from the spirit or scope of the present invention.

Each patent, patent application, and publication cited or described in the present application is hereby incorporated by reference in its entirety as if each individual patent, patent application, or publication was specifically and individually indicated to be incorporated by reference.

TABLE 1
Table 1: Lifespan analysis and statistics.

| | Mean | | 75th | Total # | |
|---|---|---|---|---|---|
| | Lifespan ± | Median | Percentile | Animals | |
| Strain | SEM (days) | (days) | (days) | Died/Total | P-value |
| FIG. 1 | | | | | |
| sid-1(qt9) Ex.rol-6 marker | 18.8 ± 0.7 | 19 | 24 | 77/104 | |
| ges-1p::cco-1HP | 23.9 ± 0.8 | 24 | 28 | 74/108 | <.0001 |
| wildtype N2 | 18.2 ± 0.5 | 17 | 21 | 64/106 | |
| rab-3p::cco-1HP | 21.5 ± 0.5 | 23 | 25 | 74/104 | <.0001 |
| sid-1(qt9) Ex.rol-6 marker | 18.6 ± .7 | 18 | 22 | 56/96 | |
| myo-3p::cco-1HP | 16.6 ± .5 | 16 | 20 | 76/100 | 0.0574 |
| sid-1(qt9) Ex.rol-6 marker | 19.8 ± 0.7 | 19 | 23 | 73/104 | |
| unc-119p::cco-1HP | 23.8 ± 0.8 | 24 | 28 | 65/104 | 0.0001 |
| FIG. 2 | | | | | |
| N2 on EV | 16.1 ± 0.4 | 16 | 20 | 91/97 | <.0001 |
| N2 on cco-1 RNAi | 23.1 ± 0.6 | 24 | 27 | 93/104 | |
| rde-1(ne219) on empty vector | 18.0 ± 0.3 | 16 | 20 | 103/113 | .4043 |
| rde-1(ne219) on cco-1 | 18.2 ± 0.4 | 18 | 21 | 96/111 | |
| Intestinal nhx-1p::rde-1 rescue on | 14.6 ± 0.6 | 15 | 17 | 85/101 | <.0001 |
| EV | | | | | |
| Intestine nhx-1p::rde-1 on cco-1 | 22.0 ± 0.2 | 23 | 27 | 77/103 | |
| RNAi | | | | | |
| Muscle hlh-1p::rde-1 rescue on EV | 13.5 ± 0.3 | 14 | 15 | 64/115 | <.0002 |
| Muscle hlh-1p::rde-1 rescue on cco- | 11.8 ± 0.3 | 12 | 13 | 51/116 | |
| 1 RNAi | | | | | |
| Hypodermis rde-1 rescue on EV | 13.4 ± 0.4 | 15 | 16 | 100/127 | 0.148 |
| Hypodermis rde-1 rescue on cco-1 | 14.3 ± 0.4 | 15 | 18 | 98/122 | |
| RNAi | | | | | |
| Intestinal nhx-1p::rde-1 rescue on | 16.3 ± 0.4 | 16 | 18 | 79/92 | |
| EV | | | | | |
| Intestinal nhx-1p::rde-1 rescue on | 16.4 ± 0.4 | 16 | 18 | 92/104 | 0.8164 |
| EV/daf-16 RNAi (50/50) | | | | | |
| Intestinal nhx-1p::rde-1 rescue on | 19.9 ± 0.6 | 18 | 24 | 51/103 | <.0001 |
| cco-1/daf-16RNAi (50/50) | | | | | |
| FIG. 4 | | | | | |
| N2 on EV | 19.0 ± 0.5 | 21 | 22 | 85/98 | 0.0834 |
| N2 on ubl-5 RNAi | 20.3 ± 0.4 | 21 | 24 | 87/103 | |
| daf-2(e1370) on EV | 40.1 ± 1.2 | 43 | 49 | 67/124 | |
| daf-2(e1370) on ubl-5 RNAi | 39.9 ± 1.2 | 42 | 50 | 58/137 | 0.3273 |
| eat-2(ad116) on EV | 26.4 ± .6 | 29 | 30 | 77/112 | |
| eat-2(ad116) on ubl-5 RNAi | 23.3 ± 7 | 23 | 29 | 75/105 | 0.0004 |
| N2 on EV | 18.2 ± 0.4 | 18 | 21 | 94/104 | |
| isp-1(qm150) on EV | 25.8 ± 1.0 | 27 | 33 | 64/126 | <.0001 |
| isp-1(qm150) on ubl-5 RNAi | 15.5 ± 0.7 | 13 | 18 | 37/94 | |

p-values were calculated for individual experiments, each consisting of control and experimental animals examined at the same time.
The total number of observations equals the number of animals that died plus the number censored.
Animals that died prematurely (exploded out the vulva, bagged, crawled off the plate) were censored at the time of the event.
Control and experimental animals were assayed and transferred to fresh plates at the same time.
The log-rank (Mantel-Cox) test was used for statistical analysis.

TABLE 2
Table 2
Percent survival was determined at 7 hours in 0.01 mM paraquat.
Mean lifespan was determined using JMP5.1 statistical analysis. See
Supplemental Materials and Methods for assay conditions.

| | % Survival at 7 hours in |
|---|---|
| | .01 mM paraquat |
| N2 | 59.5 |
| N2 on cco1 | 50.0 |
| daf-2(e1370) | 95.2 |
| mev-1(kn1) | 4.8 |
| sid-1/rol-6 | 57.1 |
| ges-1p HP | 50.0 |
| rab-3p HP | 64.3 |
| unc-119p HP | 28.6 |
| myo-3 HP | 52.3 |
| rde-1(ne219) | 54.8 |
| rde-1(ne219) cco-1 RNAi | 76.2 |
| intestinal rde-1 | 54.8 |
| intestinal rde-1 on cco-1 RNAi | 69.0 |
| muscle rde-1 | 50.0 |
| muscle rde-1 on cco-1 RNAi | 57.1 |

TABLE 3
Table 3
Mean lifespan of animals treated with 1200 J/m² of UV.
Worms were scored daily for viability. See Supplemental Materials and
Methods for assay conditions.

| | UV treated |
|---|---|
| | Mean lifespan |
| Strain | (days) |
| sid-1/rol-6 | 4.9 ± 0.21 |
| ges-1p HP | 4.9 ± 0.13 |
| unc-119p HP | 4.7 ± 0.15 |
| myo-3 HP | 3.5 ± 0.14 |

TABLE 4
Table 4
Worms were exposed to 35° C. temperature stress and assayed
every 2 hours for viability. Shown above is the percent survival at 10
hours. See Supplemental Materials and Methods for assay conditions.

| | % survival at 10 hours 35 |
|---|---|
| Strain | degrees Celsius |
| N2 | 77 |
| daf-2(e1370) | 100 |
| mev-1(kn1) | 49 |
| sid-1/rol-6 | 37 |
| ges-1p HP | 26 |
| unc-119p HP | 68 |
| myo-3 HP | 26 |

TABLE 5
Table 5

| | Mean | | |
|---|---|---|---|
| | Lifespan ± SEM | Median | 75% Failures |
| Strain | (days) | Time (days) | (days) |
| N2 on EV | 17.3 ± 0.4 | 17 | 20 |
| sid-1(qt9) on EV | 19.6 ± 0.5 | 19 | 24 |
| 37.1 sid-1/rol-6 | 18.8 ± 0.6 | 19 | 24 |
| 19.5 unc-119p::cco-1HP | 21.0 ± 0.6 | 20 | 25 |
| 53.2 ges-1p::cco-1HP | 21.5 ± 0.6 | 22 | 25 |
| 54.1 ges-1p::cco-1HP | 22.1 ± 0.8 | 22 | 25 |
| 54.3 ges-1p::cco-1HP | 22.8 ± 0.7 | 24 | 28 |
| 54.5 ges-1p::cco-1HP | 21.9 ± 0.6 | 22 | 25 |
| 55.9 ges-1p::cco-1HP | 23.9 ± 0.8 | 24 | 28 |
| A2 ges-1p::cco-1HP | 21.8 ± 0.7 | 22 | 25 |
| 65.11 myo-3p::cco-1HP | 14.4 ± 0.5 | 15 | 15 |
| 66.15 myo-3p::cco-1HP | 15.2 ± 0.5 | 15 | 17 |
| 66.2 myo-3p::cco-1HP | 15.9 ± 0.5 | 15 | 17 |
| 66.2a myo-3p::cco-1HP | 15.6 ± 0.5 | 15 | 17 |
| 66.2 myo-3p::cco-1HP | 15.7 ± 0.6 | 15 | 19 |
| 66.6 myo-3p::cco-1HP | 17.3 ± 0.8 | 15 | 17 |
| 70.11 myo-3p::cco-1HP | 14.5 ± 0.5 | 15 | 15 |
| N2 on EV | 18.2 ± 0.5 | 17 | 21 |
| N2 on cco-1 RNAi | 26.1 ± 0.6 | 27 | 30 |
| W rab-3p::cco-1HP | 21.8 ± 0.6 | 23 | 25 |
| B3 rab-3p::cco-1HP | 19.6 ± 0.8 | 19 | 25 |
| sid-1(qt9) | 17.4 ± 0.7 | 18 | 20 |
| sid-1(qt9)/rol-6 | 14.5 ± 0.5 | 13 | 18 |
| 13.14 unc-119p::cco-1HP | 20.1 ± 0.7 | 20 | 24 |
| 11.2 unc-119p::cco-1HP | 17.1 ± 1.5 | 16 | 24 |
| 13.4 unc-119p::cco-1HP | 17.2 ± 0.7 | 17 | 21 |
| 13.9 unc-119p::cco-1HP | 19.3 ± 0.6 | 20 | 23 |
| 18.1 unc-119p::cco-1HP | 18.3 ± 0.8 | 16 | 23 |
| 19.5 unc-119p::cco-1HP | 21.9 ± 0.7 | 21 | 25 |
| 22.1 unc-119p::cco-1HP | 18.4 ± 0.9 | 18 | 23 |
| 9.7 unc-119p::cco-1HP | 16.1 ± 0.6 | 18 | 20 |
| N2 | 19.7 ± 0.7 | 20 | 25 |
| sid-1(qt9)/myo-2:GFP | 20.3 ± 1.0 | 15 | 28 |
| Line 10 ges-1p::cco-1HP | 25.9 ± 0.6 | 28 | . |
| Line 2 ges-1p::cco-1HP | 28.2 ± 0.8 | 33 | . |
| Line 3 ges-1p::cco-1HP | 29.5 ± 0.8 | 30 | . |
| Line 4 ges-1p::cco-1HP | 23.5 ± 0.7 | 25 | . |
| Line 5 ges-1p::cco-1HP | 98.4 ± 0.8 | 30 | 35 |
| Line 8 ges-1p::cco-1HP | 28.0 ± 1.3 | 30 | 35 |
| Line5 ges-1p::cco-1HP | 26.3 ± 1.0 | 28 | 30 |

1. A method of increasing longevity in an animal, comprising modulating electron transport chain function in a tissue specific manner in the animal.

2. The method of claim 1, wherein electron transport chain function is modulated in intestinal tissue.

3. The method of claim 1, wherein electron transport chain function is modulated in neuronal tissue.

4. The method of claim 1, comprising modulating the activity of cco-1 or a homolog thereof.

5. A method of increasing longevity in an animal, comprising modulating the mitochondrial unfolded protein response system in the animal.

6. The method of claim 5, comprising modulating ubl-5 or a homolog thereof.

7. The method of any of claim 1, wherein a cell non-autonomous signal is produced in the animal.

8. The method of claim 1, wherein electron transport chain function is modulated by administration of an inhibitor of an electron transport chain component.

9. The method of claim 8, wherein the inhibitor is an siRNA.

10. The method of claim 8, wherein the electron transport chain component is cco-1 or a homolog thereof.

11. The method of claim 5, wherein the mitochondrial unfolded protein response system is modulated by administration of an inhibitor of a component of the unfolded protein response system.

12. The method of claim 11, wherein the component of the unfolded protein response system is ubl-5 or a homolog thereof.

13. A method of identifying a compound that modulates longevity, comprising contacting a non-human animal with a test compound; and measuring the activity of an electron transport chain (ETC) component in the presence and absence of the test compound in said non-human animal, wherein a test compound that inhibits the activity of the ETC component in the non-human animal indicates a compound that modulates longevity.

14. The method of claim 13, wherein the non-human animal is C. elegans.

15. The method of claim 13, wherein the activity of the ETC component that is inhibited is expression of the ETC component in the animal.

16. The method of claim 13, wherein the electron transport chain component is cco-1 or a homolog thereof.

17. The method of claim 16, wherein the non-human animal expresses cco-1 in a tissue-specific manner.

18. The method of claim 13, wherein the test compound enhances longevity in the non-human animal.

19. The method of claim 18, wherein the test compound inhibits expression of ceo-1 in intestinal and/or neuronal tissue of the non-human animal.

20. The method of claim 13, further comprising assessing induction of the mitochondrial unfolded protein response in the animal.

21. The method of claim 20, wherein the test compound induces the mitochondrial unfolded protein response in the non-human animal.

22. A pharmaceutically acceptable composition comprising a mitokine in an amount effective to stimulate the mitochondrial unfolded protein response in an animal in need thereof.

23. A method of enhancing longevity in an animal, comprising administering to the animal a pharmaceutically effective amount of the composition of claim 22.

24. The method of claim 23, wherein the animal is a human.

25. The method of claim 5, wherein a cell non-autonomous signal is produced in the animal.
